(19) United States
(12) Patent Application Publication
(10) Pub. No.: US 2006/0030014 A1
(43) Pub. Date: Feb. 9, 2006

(54) Synthesis of polyhydroxyalkanoates in the cytosol of yeast

(76) Inventors: Bo Zhang, Minneapolis, MN (US); Ross P. Carlson, Bozeman, MT (US); Friedrich Srienc, Lake Elmo, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/195,521

(22) Filed: Aug. 2, 2005

Related U.S. Application Data
(60) Provisional application No. 60/598,698, filed on Aug. 3, 2004.

(51) Int. Cl. C12P 7/62; C12N 9/18; C12N 1/18; C12N 15/74

(52) U.S. Cl. 435/135; 435/254.21; 435/483; 435/197

(57) ABSTRACT

Transgenic yeast strains and methods for producing polyhydroxyalkanoate (PHA). A genetically engineered Pseudomonas oleovorans polyhydroxyalkanoate (PHA) polymerase was expressed in the cytosol of some wild type yeast strains, the pex5 mutants and a fox3 mutant. The composition of the PHA was influenced by the genetic background of the yeast host, the monomer specificity of the polymerase, the cellular compartment in which the polymerase was active, and the substrate supplied in the medium. The culture strategies and further metabolic pathway engineering technologies were provided. This platform provides a basis for controlling the composition and thus the properties of the synthesized PHA.

### CROSS-REFERENCE TO RELATED APPLICATIONS

This application claims the benefit of priority to U.S. Provisional Patent Application No. 60/598,698, entitled “SYNTHESIS OF POLYHYDROXYALKANOATES IN THE CYTOSOL OF YEAST,” the entire disclosure of which is herein incorporated by reference.

### FIELD

The present invention pertains to biosynthesis of polyhydroxyalkanoate and, more particularly, to improve microbial strains useful in the production of polyhydroxyalkanoates.

### BACKGROUND

The production of plastics in the United States exceeded 22 billion kilograms in 1986, topped 27 billion kilograms in 1991, and reached 35 billion kilograms in 1997. Nearly one third of these plastics were produced for short-term disposable applications such as packaging. As a result, municipal solid waste may contain 7% plastic by weight or 18% by volume.

Most of these synthetic polymeric materials are not susceptible to biodegradation because microbes generally do not contain the enzymes needed to digest structures not occurring in nature, including most monomers in plastics and chiral monomers with the left-handed or “L” conformation. Indeed, most polymers have traditionally been designed for maximum stability.

Massive environmental and disposal problems are associated with this large scale production of plastic wastes. Landfill space is increasingly scarce, with many cities, particularly in the United States, rapidly exhausting their capacity. Potentially, hundreds of thousands of marine animals are killed annually by the estimated one million tons of plastic debris dumped into the world's oceans each year. In addition, the litter is always an aesthetic, as well as an environmental, problem. Recycling of these plastics is hindered by a limited field of applications for recycled plastics and processing difficulties, including sorting of the various types of plastics.

### BRIEF SUMMARY

The invention provides microorganisms for the production of polyhydroxyalkanoate (PHA) and improved methods for producing PHA. In at least some embodiments, the microorganisms include transgenic yeast cells. Formation of PHA in yeast may occur, for example, by way of polymerization of one or more hydroxyalkanoates and is catalyzed by a heterologous PHA polymerase. Example yeast cells may include cells of the genera Saccharomyces (e.g., S. cerevisiae), Pichia, Kluyveromyces, or any other suitable genera. Biologically synthesized PHA typically accumulates in the yeast and can be isolated. Some additional details regarding these as well as some of the other embodiments contemplated are described in more detail below.

The above summary of some embodiments is not intended to describe each disclosed embodiment or every implementation of the present invention. The Figures, Detailed Description, and Examples, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 shows the general structure of polyhydroxyalkanoates (PHA);

FIG. 2 depicts vectors for PHA polymerase (phaCL) gene expression;

FIG. 3 is a vector for E. coli acyl-CoA dehydrogenase (fadE) gene expression;

FIG. 4 shows GC-MS analysis of PHA produced by S. cerevisiae BY4743, when lauric acid (C12) was used as the carbon source;

FIG. 5 illustrates expression of PHA synthesis pathway in the cytosol of S. cerevisiae pex5 mutant;

FIG. 6 shows GC-MS analysis of PHA produced by S. cerevisiae BY4743-YDR244W, when lauric acid (C12) was used as the carbon source;

FIG. 7 shows GC-MS analysis of PHA produced by S. cerevisiae BY4743-YDR244W, when different fatty acids were used as the carbon source;

FIG. 8 shows GC-MS analysis of PHA produced by S. cerevisiae pex5-3c11 harboring p2TG1T-700, when tridecanoic acid and undecanoic acid were used as the carbon source;

FIG. 9 illustrates the effect of the different pH value to PHA content and cell dry weight (CDW) produced by S. cerevisiae BY4743-YDR244W harboring p2TG1T-700(H), when lauric acid was used as carbon source;

FIG. 10 is GC-MS analysis of PHA produced by S. cerevisiae pex5-3c11 harboring p2TG1T-700, when lauric acid was used as the carbon source;

FIG. 11 shows the construction of plasmids pDP-307 and p2DP307T;

FIG. 12 depicts vectors for sc1-PHA synthase phbC) gene expression;

FIG. 13 shows the vector for GFP gene expression in yeast;

FIG. 14 illustrates viability analysis and Gfp expression of yeast cells cultured in SO medium; and

FIG. 15 shows viability analysis and Gfp expression of yeast cells after being “boosted” in YP medium, then cultured in various media.

### DETAILED DESCRIPTION

The following description should be read with reference to the drawings wherein like reference numerals indicate like elements throughout the several views. The detailed description and drawings illustrate example embodiments of the claimed invention.

Polyhydroxyalkanoates (PHAs) are a broad class of polyesters that are formed naturally in many species of bacteria as storage materials for carbon, energy and reducing equivalents. These biological compounds have received considerable interest as renewable resource based, biodegradable, and biocompatible plastic with a wide range of potential applications. Polyhydroxyalkanoate (PHA) is a commercially useful polymer that can be completely biodegraded to carbon dioxide and water. Its properties are similar to those of polypropylene, which represented 11% of U.S. polymer production in 1986. In addition, it is human biocompatible, which makes it a useful material for medical implants.

Recently, significant research effort has focused on such issues as designing improved synthesis pathways for “smarter” PHAs which possess more desirable and valuable physical properties.

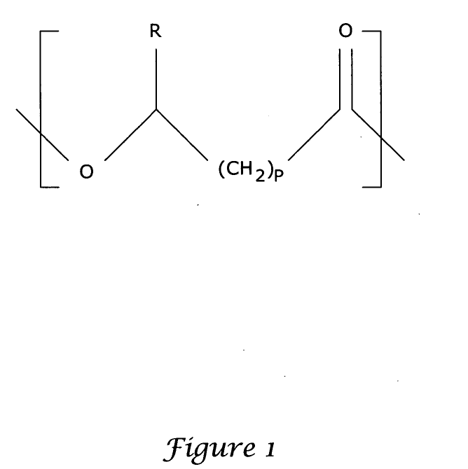

PHAs are polyesters of hydroxyalkanoates conforming to the general structure illustrated in FIG. 1. Each monomer contains a carboxyl and a hydroxyl functional group. Unless the R group is hydrogen, the adjacent carbon is a chiral center. The R groups and P values for several PHAs are listed in Table 1 below. The value of n is typically about 100 to about 30,000. More complex PHAs can contain olefin, branched, halogenated, phenyl, hydroxyl, cyclohexyl, ester, or nitrile R groups. A list of selected constituents detected in microbial PHAs is found in Steinbuchel, Biomaterials: Novel Materials from Biological Sources, pp. 123-213, p. 128, Stockton Press: New York (1991), which is incorporated herein by reference.

TABLE 1
Selected Bacterial Polyhydroxyalkanoates

| Polyhydroxyalkanoates* | R | P |
|---|---|---|
| Poly-3-hydroxypropionate* | —H | 1 |
| Poly-3-hydroxybutyrate* | —CH3 | 1 |
| Poly-3-hydroxyvalerate* | —CH2CH3 | 1 |
| Poly-3-hydroxyhexanoate | —CH2CH2CH3 | 1 |
| (or hydroxycaproate) | | |
| Poly-3-hydroxyheptanoate | —CH2CH2CH2CH3 | 1 |
| Poly-3-hydroxyoctanoate | —(CH2)4CH3 | 1 |
| Poly-3-hydroxynonanoate | —(CH2)5CH3 | 1 |
| Poly-3-hydroxydecanoate | —(CH2)6CH3 | 1 |
| Poly-3-hydroxyundecanoate | —(CH2)7CH3 | 1 |
| Poly-3-hydroxydodecanoate | —(CH2)8CH3 | 1 |
| Poly-4-hydroxybutyrate* | —H | 2 |
| Poly-4-hydroxyvalerate* | —CH3 | 2 |
| Poly-5-hydroxybutyrate* | —H | 3 |
| Poly-3-hydroxy-4-pentenoate* | —CH═CH2 | 1 |
| Poly-3-hydroxy-2-butenoate | —CH3 | 1 |
| (unsaturated chain)* | | |

*These polymers are short chain length monomer polyhydroxyalkanoates

Physiological data and enzymatic studies have shown that there are two distinct classes of PHAs: polymers formed from short chain length carbon monomers (referred to herein as scl-PHA) and polymers formed from medium chain length carbon monomers (referred to herein as mcl-PHA). A “short chain length carbon monomer” is a carbon monomer having 3 carbon atoms (a C3 monomer) to about 5 carbon atoms (a C5 monomer). Examples of short chain length carbon monomers include 3-hydroxybutyrate and 3-hydroxyvalerate, which are formed from glucose and glucose supplemented with propionic acid, as substrates, respectively, for the polymerase. A “medium chain length carbon monomer” is a carbon monomer having about 6 carbon atoms (a C6 monomer) to about 14 carbon atoms (a C14 monomer). Examples of medium chain length carbon monomers include straight-chain 3-hydroxyalkanoic acids with about 6 to about 12 carbon atoms, which are formed from the respective alkanoic monomer as substrate for the polymerase. In all, ninety-one PHA monomer units have been discovered to date.

A PHA polymerase is an enzyme that is capable of catalyzing the polymerization of constituent monomers to yield PHA, and is also referred to in scientific literature as a PHA synthase or a PHA synthetase. The term “scl-PHA polymerase,” as used herein, refers to a PHA polymerase that is capable of catalyzing the polymerization of monomers or precursors that include 3 to about 5 carbon atoms, to yield scl-PHA homopolymers or copolymers. PHB polymerase is an example scl-PHA polymerase. Biopolymers that can be synthesized with scl-PHA polymerases include PHAs such as poly(3-hydroxybutyrate) and poly(3-hydroxybutyrate-co-3-hydroxyvalerate), for example.

As used herein, “mcl-PHA polymerase” refers to a PHA polymerase that is capable of catalyzing the polymerization of monomers or precursors that include about 6 to about 14 carbon atoms, to yield mcl-PHA homopolymers or copolymers. Biopolymers synthesized with mcl-PHA polymerases include poly(3-hydroxyoctanoate) (PHO), poly(3-hydroxyhexanoate) (PHH), and poly(3-hydroxydecaonoate), for example.

PHA polymerases may be naturally occurring or non-naturally occurring. A non-naturally occurring PHA polymerase includes a naturally occurring polymerase that has been modified using any technique that results in addition, deletion, modification, or mutation of one or more amino acids in the enzyme polypeptide sequence, such as by way of genetic engineering, as long the catalytic activity of the enzyme is not eliminated. For example, a polymerase according to the present invention can include an N-terminal or C- terminal amino acid sequence that directs or targets the enzyme. The PHA polymerase activity can be part of a bifunctional or multifunctional enzyme or enzyme complex; thus the term PHA polymerase is intended to include such bifunctional or multifunctional enzymes that possess PHA polymerase activity.

The present invention relates to the expression of heterologous genes involved in the synthesis pathway of polyhydroxyalkanoate biopolymers in transgenic yeast cells. A “heterologous” nucleic acid fragment, or gene, is one containing a nucleotide sequence that is not normally present in the cell, for example a prokaryotic nucleotide sequence that is present in a eukaryotic cell. A heterologous gene is also referred to herein as a transgene. As used herein, “transgenic” refers to an organism in which a nucleic acid fragment containing a heterologous nucleotide sequence has been introduced. The transgenes in the transgenic organism are preferably stable and inheritable. The heterologous nucleic acid fragment may or may not be integrated into the host genome.

The term “yeast” is used herein to refer to any yeast that can be genetically transformed, including but not limited to the genera Saccharomyces (e.g., S. cerevisiae), Pichia, Kluyveromyces, and the like.

The transgenic yeast can be cultured in any convenient matter, for example in a suspension or on a solid matrix. Microbial cultures are typically grown in a nutrient-rich culture medium. The transgenic cells of the invention can be grown under aerobic condition.

Yeasts of the invention are transformed with a nucleic acid fragment comprising a heterologous nucleotide sequence and, preferably, but not necessarily, regulatory sequences operably linked thereto. The nucleic acid fragment can be circular or linear, single-stranded or double stranded, and can be DNA, RNA, or any modification or combination thereof. Typically a vector comprising the heterologous nucleotide sequence is used for transformation. The vector can be a plasmid (integrative or autonomous), a viral vector, a cosmid, or any other suitable vector. Selection of a vector backbone depends upon a variety of desired characteristics in the resulting construct, such as a selection marker, plasmid reproduction rate, and the like.

Some example yeasts are transformed with a heterologous nucleotide sequence that encodes a functional PHA polymerase and may, in some embodiments, optionally be transformed with one or more additional heterologous nucleotide sequences that encode at least one other functional enzyme utilized in the biosynthesis of PHA such as acyl-CoA oxidase and/or trans-2-enoyl-CoA hydratase II. Yeasts that are transformed to produce PHA can be further transformed to express or overexpress acyl-CoA synthetase. Different combinations of genes can be expressed.

Some other example yeasts are wild type yeasts and the yeast strain comprises a pex5, pex7, pex8, pex13, pex14, pex18, pex21 and/or fox3 mutation. Yeasts can be further modified, such as knocking out other pex, fox and/or fatty acids synthesis pathway genes.

The S. cerevisiae pex5 mutant is viable but accumulates peroxisomal, leaflet-like membrane structures and is deficient in the import of peroxisomal matrix enzymes with a SKL-like import signal such as Fox2p. The acyl-CoA oxidase, Fox1p, follows a novel, non-PTS1 (Type 1 peroxisomal targeting sequence), import pathway that is also dependent on Pex5p. In pex5 mutants, both Fox2p and Fox1p are found in the cytosol, but Fox3p is located in the peroxisome. Activation of fatty acids entering S. cerevisiae can be mediated by at least four different acyl-CoA synthetase gene products. One of these enzymes, Faa2p, is a peroxisomal protein which carries a PTS1 like targeting sequence, while the other three enzymes do not show any obvious peroxisomal targeting sequences. A pex5 mutant is expected to retain the Faa2p in the cytosol enabling cytosolic fatty acid activation. A transgenic pex5 mutant is able to produce PHA in the cytosol.

One or more nucleic acid fragments can be used to transform a host cell. For example, the yeast can be transformed with one vector comprising a heterologous nucleic acid that encodes a PHA polymerase, and a second vector comprising a heterologous nucleic acid that encodes an acyl-CoA oxidase. Alternatively, two or more heterologous nucleic acids can be present on the same nucleic acid fragment used to transform the host cell, as is the case, for example, when a divergent promoter is used. The PHA polymerase can be a scl-PHA polymerase or a mcl-PHA polymerase. The nucleic acid sequence encoding mcl-PHA polymerase may be derived from Pseudomonas oleovorans. Nucleic acid sequences encoding scl-PHA polymerase may be derived from R. eutropha. Nucleotide sequences for these and other suitable genes are readily available to one of skill in the art from protein and nucleic acid databases such as GENBANK.

The nucleic acid fragment used to transform the yeast can optionally include a promoter sequence operably linked to the nucleotide sequence encoding the enzyme to be expressed in the host. A promoter is a DNA fragment that can cause transcription of genetic material. Transcription is the formation of an RNA chain in accordance with the genetic information contained in the DNA. The invention is not limited by the use of any particular promoter, and a wide variety are known. Promoters act as regulatory signals that bind RNA polymerase in a cell to initiate transcription of a downstream (3′ direction) coding sequence. A promoter is “operably linked” to a nucleotide sequence, if it does, or can be used to control or regulate transcription of that nucleotide sequence. The promoter used can be a constitutive or an inducible promoter. It can be, but need not be, heterologous with respect to the host.

A divergent promoter can also be used to introduce and regulate multiple genes. These promoters permit the co-regulation of two separate genes from a single, centrally located sequence. Examples of divergent promoters include the GAL1-10 promoter. Galactose inducible promoters GAL1, GAL7, and GAL10 are useful for high-level expression of both homologous and heterologous genes. The galactose metabolic pathway, from which the GAL 1, GAL7, and GAL10 promoters originate, can be regulated at the gene expression level by the regulatory proteins GAL4 and GAL80.

The heterologous nucleotide sequence can, optionally, include a start site (e.g., the codon ATG) to initiate translation of nucleic acid to produce the enzyme. It can, also optionally, include a termination sequence to end translation. A termination sequence is typically a codon for which there exists no corresponding aminoacetyl-tRNA, thus ending polypeptide synthesis. The heterologous nucleotide sequence can optionally further include a transcription termination sequence.

The nucleic acid fragment used to transform a yeast cell of the invention may optionally include one or more marker sequences, which typically encode a gene product, usually an enzyme, which inactivates or otherwise detects or is detected by a compound in the growth medium. For example, the inclusion of a marker sequence can render the transgenic cell resistant to an antibiotic, or it can confer compound-specific metabolism on the transgenic cell. Examples are marker sequences that confer kanamycin, ampicillin or paromomycin sulfate resistance; the URA3 selection marker and HIS3 selection markers described in the following examples, or, for yeast, various other genes that complement auxotrophic mutations such as G418.

A transgenic yeast of the invention can include a first heterologous nucleotide sequence encoding a PHA polymerase, and, optionally, either or both of a second heterologous nucleotide sequence encoding an acyl-CoA oxidase and a third heterologous nucleotide sequence encoding a trans-2-enoyl-CoA hydratase II reductase. One strategy for introducing multiple genes is to clone multiple promoters and genes on a single plasmid. Multiple genes can also be introduced using multiple distinct plasmids. In order to maintain the recombinant DNA, a different selection marker would be required for each plasmid. Integration or autonomous vectors can be used in introducing multiple genes into a host.

In yeast, the heterologous nucleotide sequence can be targeted to a peroxisome, one of sites of PHA precursors. Peroxisomal targeting sequences have been found on the C-terminal of several peroxisomal proteins. Peroxisomal targeting sequences having the so-called “SKL motif” have been found to be an evolutionarily-conserved transit peptide targeting expression to the peroxisomes of mammals, insects, plants and yeast. The SKL motif comprises serine, alanine or cysteine at the first position; lysine. histidine or arginine at the second position; and leucine at the third position. This sequence has been found to be effective even with folded or multiunit proteins. A detailed review of peroxisomal targeting sequences can be found in U.S. Pat. No. 6,103,956 (Srienc et al.), the entire disclosure of which is herein incorporated by reference.

In some embodiments, the heterologous nucleotide sequence includes, within the region that encodes the enzyme to be expressed, a nucleotide sequence that encodes an amino acid sequence or motif that directs the enzyme to a yeast peroxisome.

The heterologous nucleotide sequence described above can be introduced into the yeast using a variety of techniques. Transformation is preferably accomplished using electroporation. or chemical methods such as those that utilize a surfactant and/or a divalent cationic salt such as CaCl₂ or LiCl₂.

The forgoing discussion provides a basis for controlling the composition and thus the properties of the synthesized PHA. For example, polymers of even, odd, or a combination of even and odd numbered monomers can be controlled by feeding the appropriate substrates like fatty acids and glycerol. In addition, the distribution of the monomers can also be influenced by feeding substrates like pyruvate and acetate along with a fatty acid. The presented strategies all hold the potential of creating polymers with novel and desirable material properties.

PHAs were synthesized in either the cytosol or the peroxisome from intermediates of the fatty acid metabolism. The composition of the PHA was influenced by the genetic background of the yeast host, the monomer specificity of the polymerase, the cellular compartment in which the polymerase was active, and the substrate supplied in the medium. The invention provides a basis for controlling the composition and thus the properties of the synthesized PHA.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

### EXAMPLES

The invention may be further clarified by reference to the following Examples, which serve to exemplify some of the preferred embodiments, and not to limit the invention in any way.

### Example 1

Vectors Constructions for Introducing PHA Genes into Yeast

S. cerevisiae Expression Systems

Recombinant DNA is typically introduced into a host using either an integrative or an autonomous plasmid. Integrative plasmids are DNA sequences that incorporate into a host's chromosome, typically through a homologous recombination event. This event occurs between a targeting sequence on the plasmid and a homologous, host chromosomal sequence. The homologous sequence used to target the integration can be a unique or a nonunique sequence. A unique targeting sequence permits only a single copy of the transforming DNA to be integrated. This approach has been used to introduce recombinant genes as well as create mutants by interrupting certain genes. Integrative plasmids can also be targeted for non-unique sequences. Such plasmids have multiple potential integration sites and a single transformation can result in numerous copies being incorporated into the chromosome.

In addition to integrative plasmids, autonomously replicating plasmids are routinely used to deliver recombinant DNA. These sequences that replicate independently of the chromosome are normally relatively small, circular pieces of DNA, however linear plasmids have also been developed. Unlike integrated plasmids, autonomous plasmids must direct their own replication and their own segregation. These functions are necessary to ensure that the mother and daughter cell both retain the plasmid after cell division. In addition to using autonomous plasmids and integrated genes separately, the two systems can be combined.

DNA replication sequences used in plasmid expression systems in yeast can be divided into two categories: those that are based on yeast chromosomal DNA sequences and those that are based on the endogenous 2-micron circle.

Autonomously replicating sequences (ARS) are based on chromosomal DNA fragments. These sequences through a complex process initiate plasmid DNA replication and have been used to achieve high frequencies of transformation in yeast. Plasmids have been constructed which combine the ARS sequence with a centromeric DNA sequence (CEN). The CEN sequence is believed to serve as an attachment point for spindle fibers during cell division.

The 2 μm origin of replication is the most popular means of maintaining a fairly stable, high copy number plasmid. This origin of replication is derived from the endogenous S. cerevisiae 2 μm circle. This native yeast plasmid is found in numerous laboratory yeast strains. The 6.3 kb plasmid, which confers no selective advantage to its host, seems to serve no purpose other than self propagation. Different pieces of the 2 μm circle have been used to regulate the replication and segregation of expression vectors. A common piece is the 2.2 kb EcoRI fragment that in [cir+] strains of S. cerevisiae maintains between 10 and 40 plasmid copies per cell. Although 2 μm based plasmids are not as stable as CEN based plasmids, the high copy number makes these plasmids useful when high expression levels are desired.

DNA transformation systems usually employ selection markers for two purposes. First, selection markers permit the isolation of recombinant organisms after a transformation and secondly selection markers help ensure the recombinant population maintains the transforming DNA during culturing. Typical yeast selection markers are designed to complement auxotrophic host mutations. Common selection markers include genes that complement mutations involved in the synthesis of metabolites like adenine, histidine, leucine, lysine, tryptophan, or uracil. Although not as common, some yeast selection markers impart resistance to broad spectrum antibiotics such as G418.

S. cerevisiae promoters can be placed under one of two broad classifications, either constitutive or inducible. Constitutive promoters continuously direct gene expression and are typically found regulating widely utilized genes like those from glycolysis. When a gene is only required under certain environmental conditions, its expression is usually regulated by an inducible promoter. For example, the S. cerevisiae genes involved in the metabolism of galactose are regulated by a well-studied inducible promoter system.

For effective high-level expression in S. cerevisiae, mRNA termination sequences are often required. mRNA stability is thought to be a function of its nucleotide sequence, so it is advantageous to keep the mRNA molecule as small as possible to avoid any unnecessary destabilizing sequences.

E. coli Plasmid Construction

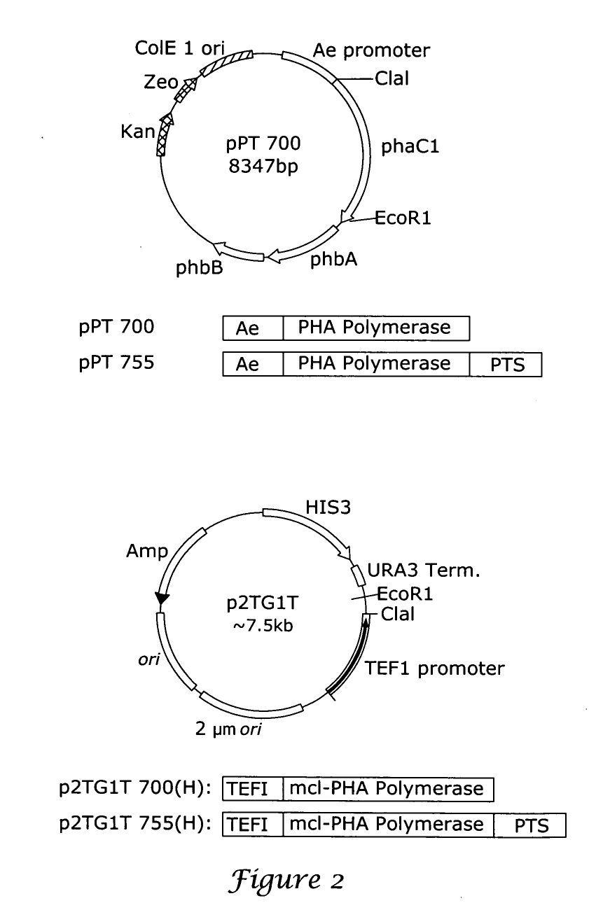

The plasmid pPT700 (FIG. 2), a vector containing the phaC1 gene isolated from Pseudomonas oleovorans and phaB, phaA genes from Ralstonia eutropha, was made as described in Jackson, Recombinant Modulation of the phbCAB Operon Copy Number in Ralstonia eutropha and Modification of the Precursor Selectivity of the Pseudomonas oleovorans Polymerase I. Masters Dissertation. University of Minnesota. St. Paul, Minn., (1998).

A peroxisomal targeting sequence (PTS) was added to pPT700 to form another plasmid pPT755. The plasmid pPT755 was constructed as follows: the phaC1 gene was obtained by PCR-cloning of pPT700. The primers used were:

SEQ ID NO.1

5′-ATTATCGATGAGTAACAAGAACAACGATGAG-3′
and

SEQ ID NO.2

5′-GGAATTCATAGCTTGGAACGCTCGTGAACGTAGG-3′

which give a ClaI upstream and an EcoRI downstream restriction site. The 3′ primer modified the phaC1 gene by the addition of a triple amino acid peptide (SKL) to the 3′ end. This type I peroxisomal targeting sequence (-SKL-COOH, PTS1) was targets expression of malate dehydrogenase (MDH3) to the peroxisomes in Saccharomyces cerevisiae. The PCR product was digested with ClaI and EcoRI, and ligated into a similarly digested pPT700 to create pPT755.

S. cerevisiae Plasmid Construction

The plasmid p2TG1T-700(H) (FIG. 2) was constructed from the plasmid p2TG1T(H) that contains the 2 μm origin of replication, HIS3 marker, TEF1 promoter and the URA3 termination sequence. The P. oleovorans mc1-PHA polymerase gene (phaC1) was isolated from the plasmid pPT700 (FIG. 2) using a ClaI and EcoRI digest and was ligated into a similarly digested p2TG1T(H). The P. oleovorans mcl-PHA polymerase gene (phaC1) containing the PTS1 peroxisomal targeting sequence was obtained from the plasmid pPT755 using a ClaI and EcoRI digest and was ligated into a similarly digested p2TG1T(H) to create p2TG1T-755(H).

### Example 2

General Materials and Methods for Production of PHA in S. cerevisiae

Unless otherwise noted, all chemicals were purchased from Sigma Chemical Company (St. Louis, Mo.) or Fisher Scientific (Fair Lawn, N.J.).

Strains

Plasmids were routinely grown in Escherichia coli strain DH5α (Life TechnologiesTM, Gaithersburg, Md.). E. coli β-oxidation defective strains are provided by the E. coli Genetic Stock Center (Yale University, New Haven, Conn.).

The Saccharomyces cerevisiae strains used are listed in following Table 2. S. cerevisiae BY4743, BY4741-YIL160C and BY4743-YDR244W (which is a pex5 heterozygous strain) were obtained from Invitrogen (Carlsbad, Calif.). The strains wt-16-4 and pex5-16-2 were sporulated from BY4743-YDR244W, and pex5-3c11 was made by mating two pex5 haploid strains according to standard protocols (F. Sherman, Methods Enzymol, 350, 3-41 (2002)). S. cerevisiae strains harboring the PHA synthase gene were maintained in SD media (0.67% yeast nitrogen base without amino acids, 2% glucose, and amino acids).

TABLE 2
List of Saccharomyces cerevisiae strains

| Name | Genotype | Origin of Strain |
|---|---|---|
| D603 | Mata/α ura3-52 lys2-801 met his3 ade2-101 reg1- | Carlson et al. |
| | 501 | (2002)^{a} and Leaf |
| | | et al. (1996)^{b} |
| YPH499 | Mata, ura3-52, lys 2-80, ade2-101, trp1-Δ63, his3- | this invention |
| | Δ200, leu2-Δ1 | |
| YPH500 | Matα, ura3-52, lys 2-80, ade2-101, trp1-Δ63, his3- | this invention |
| | Δ200, leu2-Δ1 | |
| BY4743 | Mata/α his3Δ1 leu2Δ0 ura3Δ0 | Cat. #95400- |
| | | BY4743; |
| | | Invitrogen |
| BY4743-YDR244W | Mata/α his3Δ1 leu2Δ0 ura3Δ0 pex5::kanMX4 | Cat. #95400- |
| | | 23603; |
| | | Invitrogen |
| BY4741-YIL160C | Mata his3Δ1 leu2Δ0 ura3Δ0 met15Δ0 | Cat. #95400-2319; |
| | fox3::kanMX4 | Invitrogen |
| pex5-3c11 | Mata/α his3Δ1 leu2Δ0 ura3Δ0 pex5::kanMX4 | this invention |
| pex5-16-2 | Mata his3Δ1 leu2Δ0 ura3Δ0 lys2Δ0 met15Δ0 | this invention |
| | pex5::kanMX4 | |
| wt-16-4 | Matα his3Δ1 leu2Δ0 ura3Δ0 lys2Δ0 | this invention |

^{a}Carlson et al., “Metabolic pathway analysis of a recombinant yeast for rational strain development,” Biotechnol Bioeng, 79, 121-134 (2002).
^{b}Leaf et al., “Saccharomyces cerevisiae expressing bacterial polyhydroxybutyrate synthase produces poly-3-hydroxybutyrate,” Microbiology (Reading, England), 142 (Pt 5), 1169-1180(1996).

Bacterial Growth Media

E. coli was routinely grown in LB medium (10 g/L Bacto tryptone (Difco, Detroit, Mich.), 5 g/L Bacto yeast extract (Difco), 10 g/L NaCl) or 2×YT medium(16 g/L Bacto tryptone, 10 g/L Bacto yeast extract, 5 g/L NaCl). When using Zeo gene as the screening marker, transformed E. coli was grown in Low Salt LB medium, supplemented with Zeocin (25 μg/ml). Low Salt LB medium contained 10 g tryptone, 5 g yeast extract and 5 g NaCl per liter, pH 7.5. Addition of fatty acid aided production of PHA. When appropriate, either ampicillin or kanamycin was added. E. coli cultures were normally incubated at 30° C. or 37° C.

Wild type S. cerevisiae cultures were grown on YPAD media (10 g/L Bactro yeast extract, 20 g/L Bactro peptone, 20 g/L glucose, 40 mg/L Adenine sulfate). The adenine is added to inhibit the reversion of ade1 and ade2 mutants. Transgenic yeast strains were grown on SD minimal media (6.7 g/L Bactro Yeast Nitrogen Base w/o amino acids, 10-20 g/L D-glucose). The following additions were made to complement the auxotrophic mutation of S. cerevisiae BY4743: 20 mg/L methionine, 20 mg/L leucine, and 20 mg/L histidine. To avoid problems associated with the heat stability of some species, all media components were filter sterilized (Supor-200 filter disc, pore size 0.2 μm, Gelman Sciences, Ann Arbor, Mich.). For shake flask and bioreactor experiments, enriched SD minimum medium was used. This medium resulted in a higher final biomass than the standard SD media. Modifications to previously described media include: 100 mg/L adenine, 100 mg/L methionine, 150 mg/L lysine, and 80 mg/L histidine.

For PHA production, a stationary-phase culture grown on glucose was harvested by centrifugation and cells were washed once in water and resuspended at a 1:10 dilution in fresh SOG1 media containing 0.67% yeast nitrogen base without amino acids, 1% glycerol, 0.4% Tween 80 and the appropriate fatty acids. When cultivating pex5 mutants, cultures were supplemented with geneticin. The cultures were then grown on the SOG1 media for 5-6 days before being harvested for PHA analysis. The media utilized either a 5 mM phosphate or 5 mM citrate acid buffer to control pH from 4.5 to 7.0.

Shake Flask Cultures

During shake flask studies, all experimental conditions were run in triplicate. The cultures were grown in 250 ml Erlenmeyer flasks containing 50 ml medium. The shaker was operated at 200 rpm and 30° C. All reported data is an average of the three separate flask cultures.

PHA Detection Measures

The presence and concentration of PHA in E. coli and yeast cell samples was analyzed via a number of methods. Staining granules with Nile red is a standard method of detecting PHA. Gas chromatography and gas chromatography-mass spectrometry provided evidence that a hydroxyalkanoic derivative was present and quantified it but could not determine whether or not it was polymeric.

Nile Red Staining

Nile red is a stain commonly used to detect PHA granules in bacteria. It stains lipids, including PHA, and is membrane-soluble. Bacterial cell samples. were centrifuged, and the supernatant was discarded. Cells were diluted in 150 μl ddH2O, and 7 μl of a Nile red stock solution (50 mg/ml in acetone) (Fisher) was added. After mixing, the samples were incubated at room temperature for five minutes. The stained cells were viewed under a microscope equipped with an ultraviolet lamp for detection of Nile red fluorescence at 488 nm.

Gas Chromatography

Samples for gas chromatography (GC) were prepared by propanolysis. Wet cell matter from the pellets of settling volume determinations was weighed into screw top glass test tubes, washed with 3 to 5 ml of acetone, and dried overnight. Then 0.5 ml of 1, 2-dichloroethane (Fisher), 0.5 mL of acidified propanol solution containing 20% HCl (Fisher) and 80% 1-propanol (Fisher), and 50 μL of 2 mg/mL benzoic acid (Sigma) internal standard were added. The tubes were sealed and heated in a boiling water bath for 2 to 3 hours. After the tubes had cooled to room temperature, 1 mL of deionized water was added to each tube for PHA extraction. The tubes were thoroughly mixed, and the resulting organic phase was transferred to injection bottles for GC analysis

The samples were injected into a Hewlett Packard 5890A Gas Chromatograph equipped with a Hewlett Packard 7673A automatic injector. A fused silica capillary column, DB-WAX 30W, with a length of 30 m and a 0.05 μm film thickness (J&W Scientific) was employed, and separated components were detected by a flame ionization detector. The temperature profile used was 60° C. for 0.5 minutes, increasing at a rate of 10° C. per minute for 15 minutes and 210° C. for 15 minutes.

The PHA content in the sample vials was determined by calculating the quotient (Q) of the area of the PHA peak divided by the area of the benzoic acid peak and comparing the result with Q values from a series of PHA standard solutions.

Gas Chromatography-Mass Spectrometry

Samples were prepared for gas chromatography-mass spectrometry (GC-MS) as described in the preceding subsection. Samples were injected into a gas chromatograph-mass spectrometer equipped with a DB-WAX column. GC-MS provided gas chromatographic spectra similar to those produced by GC alone. During the acidified propanolysis preparation described above, PHA is broken up into its constituent monomers, which each form an ester with propanol. In mass spectrometry, the resulting molecules are vaporized and fragmented, and the resulting patterns of ion fragments form a fingerprint by which the molecule may be identified. Masses 131, which represent the loss of a methyl group, and 87, which represents the loss of the propanoyl group, were used as diagnostic peaks (Table 3).

TABLE 3
PHA Fragment Masses

| Fragment Structure | Mass |
|---|---|
| —CH(OH)CH₂C(O)OCH₂ CH₂ CH₂ | 131 |
| —C(O)CH₂ CH(OH)CH₃ | 87 |

Nuclear Magnetic Resonance Spectroscopy

To verify the presence of polymer rather than just its constituent monomer or another hydroxyalkanoate derivative, proton nuclear magnetic resonance spectrometry (1H-NMR) was employed. Samples of cells grown in between 0.5 liter and 3 liters of shake flask culture were weighed and lyophilized. PHA was extracted from cells by refluxing for two days with chloroform in a Soxhlet extraction apparatus (Kimex). The resulting chloroform solution was evaporated and the residue resuspended in a 2.5 mL of chloroform and diluted to 12.5 mL with methanol to form a 1:5 chloroform:methanol solution. After allowing precipitate to form for twenty-four hours, the solution was centrifuged at 4,000× g for 15 minutes. The decanted pellet was washed gently in methanol and resuspended in 0.75 mL of deuterated chloroform (Sigma). The samples were then transferred to deuterated-chloroform rinsed NMR tubes and analyzed with a 300 MHz Nicolet NT -300WB Ff-NMR.

### Example 3

Novel Synthesis Routes for Polyhydroxyalkanoic Acids with Unique Properties

PHAs have attracted considerable interest as a natural, biodegradable and biocompatible plastic with the potential to be produced economically by microbial cultivation or by other biological systems. Recently, significant research effort has focused on such issues as designing improved synthesis pathways for ‘smarter’ PHAs which possess more desirable and valuable physical properties.

Physiological data and enzymatic studies have shown that there are two distinct classes of PHAs. The two distinct classes are based on the number of carbon atoms in the monomer unit. Scl-PHA (short chain length) polymers possess 3-5 carbon monomers (C3-C5), whereas mcl-PHA (medium chain length) polymers possess 6-14 carbon monomers (C6-C14). We have previously shown that expression of a bacterial PHA polymerase in the cytosol of Saccharomyces cerevisiae leads to the formation of poly (R)-3-hydroxybutyric acid (PHB). We have extended this work by expressing in this yeast a polymerase capable of polymerizing medium chain length (R)-3-hydroxy precursor molecules (mcl-PHA). We demonstrate that these engineered yeasts are capable of synthesizing mcl-PHA consisting of 6-13 carbon monomers (C6-C 13) in the cytosol. The metabolites which serve as the mcl-PHA monomers are typically produced via the β-oxidation pathway in specialized organelles known as peroxisomes. Therefore, the results indicate that the β-oxidation pathway is not restricted to peroxisomes but also appears to be functional in the yeast cytosol. This finding provides a basis for novel metabolic engineering strategies that could make the PHA synthesis process more economical and could yield polymers with unique material properties.

Materials and Methods

Strains and Media

All plasmids were maintained and propagated in Escherichia coli DH5α. Saccharomyces cerevisiae strain BY4743 (Mata/α his3Δ1 leu2Δ0 ura3Δ0) was obtained from Invitrogen. S. cerevisiae harboring a PHA synthase plasmid was maintained in SD media (0.67% yeast nitrogen base without amino acids, 2% glucose, and amino acids). For PHA production, a stationary-phase culture was harvested by centrifugation. The cells were washed once in water and resuspended at a 1:10 dilution in fresh SOG1 media containing 0.67% yeast nitrogen base without amino acids, 1% glycerol, 0.4% Tween 80 and fatty acids. Cells were then cultured for an additional 5-6 days before harvesting the cells for PHA analysis. The pH was maintained at 5 with a 5mM citric acid buffer.

Cloning Procedure

The plasmids p2TG1T-700(H) and p2TG1T-755(H) are described in Example 1 and depicted in FIG. 2.

Analysis of PHA

The cytosolic PHA was studied using gas chromatography-mass spectroscopy analysis, which is described in Example 2.

Results

Expression of the P. oleovorans PHA Polymerase in the Cytosol of Yeast

In this Example, the P. oleovorans PHA polymerase is expressed in the cytosol of S. cerevisiae BY4743. The plasmid p2TG1T-700 contains the high copy number yeast 2 μm origin of replication and the HIS3 selection marker. The PHA polymerase is under the control of the constitutive TEF1 promoter and URA3 transcription termination sequence. Plasmid p2TG1T-755(H) is identical to p2TG1T-700(H) except the P. oleovorans polymerase is modified to contain the previously described type I peroxisomal targeting sequence.

Production of Medium Chain Length (MCL)-PHA

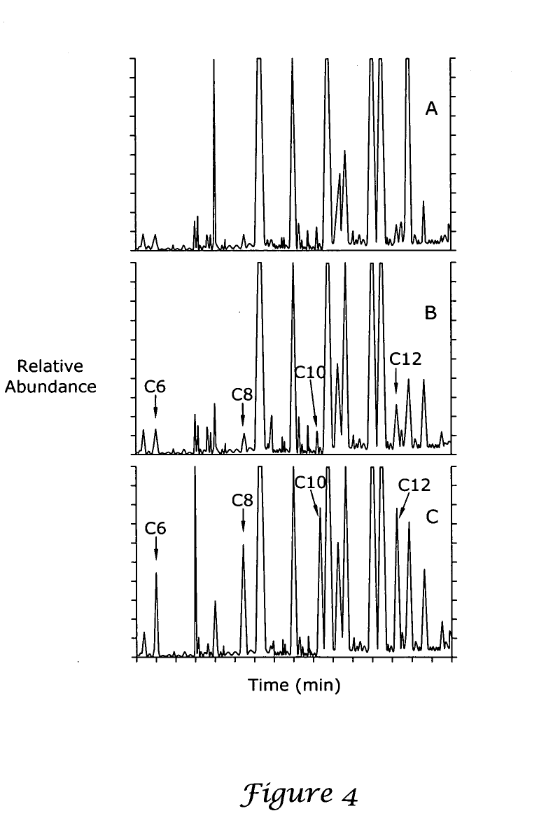

The recombinant yeasts were grown as described in the Materials and Methods, and lauric acid (C12) was used as the carbon source. The cytosolic expression of the mcl-PHA polymerase resulted in the production of PHA which accumulated to approximately 0.014% of the total cell dry weight (CDW). FIG. 4 shows GC-MS analysis of PHA produced by S. cerevisiae BY4743, when lauric acid (C12) was used as the carbon source. Only peaks, which possess a mass-to-charge ratio value of 131, are shown. FIG. 4A shows the GC-MS analysis of Wild-type S. cerevisiae BY4743. FIG. 4B shows the GC-MS analysis of S. cerevisiae BY4743 harboring plasmid p2TG1T-700(H). The C12 PHA (poly 3-hydroxydodecanoic acid) peak, C10 (poly 3-hydroxydecanoic acid), C8 (poly 3-hydroxyoctanoic acid) and C6 (poly 3-hydroxyhexanoic acid) PHA peaks are all clearly visible. Mass to charge ratios of all peaks were compared to PHA produced by E. coli harboring P. oleovorans PHA polymerase. The peroxisomally targeted PHA polymerase strain (BY4743/p2TG1T-755(H)) was used as a positive control. Under the same conditions, this strain accumulated MCL-PHA up to 0.054% of the CDW in the peroxisomes (FIG. 4C and Table 4).

TABLE 4
PHA content and monomer composition produced by
S. cerevisiae BY4743, when even-number fatty acids
were used as the carbon source.

| | | | Composition of PHA | | | |
|---|---|---|---|---|---|---|
| Carbon | | PHA content | (%, w/w) | | | |
| source | Plasmid | (% of CDW) | C12 | C10 | C8 | C6 |
| Lauric acid | p2TG1T-700 | 0.0147 ± 0.0011 | 58.6 | 16.6 | 22.9 | 1.9 |
| (C12) | | | | | | |
| Lauric acid | p2TG1T-755 | 0.0539 ± 0.0041 | 38.7 | 23.8 | 29.9 | 7.6 |
| (C12) | | | | | | |
| Oleic acid | p2TG1T-700 | Not detected | nd | nd | nd | nd |
| (C18) | | | | | | |
| Oleic acid | p2TG1T-755 | 0.0385 ± 0.0076 | 47.1 | 23.2 | 23.9 | 6.7 |
| (C18) | | | | | | |

nd: not detected

Composition of MCL-PHA Produced in the Cytosol of Yeast

In order to determine the influence of the carbon source on PHA monomer composition, the recombinant yeast were grown in SOG1 media containing one of the following fatty acids: oleic acid, tridecanoic acid (C13), lauric acid (C12) and undecanoic acid (C11). Tables 4 and 5 show that the accumulated PHA composition is dependent on the nature of the externally fed fatty acids. When lauric acid (C12) was used as the carbon source, C12 PHA is the major component of the PHA. About 58% of total PHA was comprised of C12 monomer while no C14 PHA was detected (Table 4). In yeast BY4743 harboring plasmid p2TG1T-755(H), lauric acid was presumably degraded in the peroxisomes and significant amounts of C10-C6 monomers were incorporated into the PHA by the peroxisomally targeted MCL-PHA polymerase.

Similarly, recombinant yeast grown on tridecanoic acid (C13) and undecanoic acid (C11) produced PHA containing odd-chain monomers ranging from C13 to C7 with the major components being C13 and C11 monomers (Table 5). When the yeast were grown on oleic acid (C18), no PHA was detected in the strain expressing the cytosolic polymerase, however the yeast strain with the mcl-PHA polymerase targeted to the peroxisomes accumulated PHA to approximately 0.0385% of its CDW (Table 4).

TABLE 5
PHA content and PHA monomer composition of polyester
produced by S. cerevisiae BY4743 harboring plasmid
p2TG1T-700(H) when different odd-number fatty acids
were used as the carbon source.

| | | Composition of PHA | | | |
|---|---|---|---|---|---|
| | PHA content | (%, w/w) | | | |
| Carbon source | (% of CDW) | C13 | C11 | C9 | C7 |
| Tridecanoic acid (C13) | 0.0498 ± 0.0117 | 24.2 | 16.1 | 37.6 | 21.9 |
| Undecanoic acid (C11) | 0.0255 ± 0.0048 | nd | 50.9 | 46.5 | 2.6 |

nd: not detected

Discussion

The yeast strain cytosolically expressing the PHA polymerase did not produce PHA from oleic acid (C18). However, PHA was produced from oleic acid in the strain which expressed a peroxisomally targeted PHA polymerase. These results suggest that the β-oxidation intermediates do not transverse the peroxisome membrane and that the nontargeted mcl-PHA polymerase is not transported into the peroxisomes.

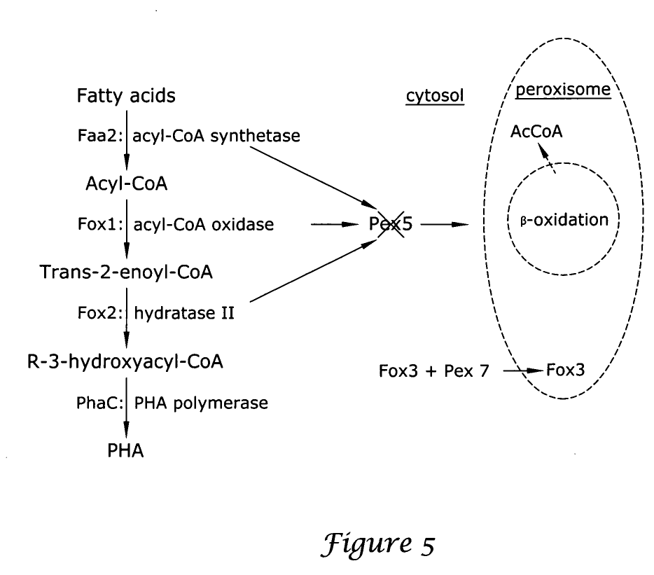

Based on the observation that the recombinant yeast expressing a cytosolic polymerase accumulate PHA monomers with C-backbones of different lengths than the fed fatty acids, we propose that β-oxidation can occur, at least partially, in the cytosol of S. cerevisiae (FIG. 5). One possible explanation for this observation is that β-oxidation enzymes are synthesized in the cytosol and then transported into the peroxisomes posttranslationally. This creates a temporal window where they could be active in the cytosol. In fact, some studies have shown that 15-25% of α-oxidation enzyme activities can be found in the cytosol of yeast. Another potential source of PHA precursors is from fatty acid biosynthesis. Both externally fed fatty acids and fatty acid biosynthesis may contribute to the observed cytosolic mcl-PHA synthesis.

### Example 4

Production of PHA in Yeast pex5 Mutants

It has been previously shown that poly β-hydroxybutyrate (PHB) is synthesized in the cytosol of S. cerevisiae if the scl-PHA polymerase from Ralstonia eutropha is expressed in this cell compartment. This finding indicates that native S. cerevisiae is capable of synthesizing monomers of the correct enantiomeric configuration for the polymerase enzyme. We have recently shown that mcl-PHA can be synthesized in the cytosol if the mcl-PHA polymerase from Pseudomonas oleovorans is expressed in S. cerevisiae (Example 3) and hypothesized that mcl-PHA precursors are likely made based on peroxisomal enzymes that remain in the cytoplasm.

To synthesize mcl-PHA in the cytosol of S. cerevisiae based on β-oxidation intermediates, key peroxisomal proteins, including Faa2p, Fox1p, and Fox2p must be active in the cytosol together with PHA polymerase (FIG. 5). Enzymes destined to the peroxisomal matrix are imported from the cytosol in a process involving specific targeting signals. Two different signals have been identified which are believed to be sufficient for transporting proteins into the peroxisome. One is the C-terminal peroxisomal targeting signal 1(PTS1) that is present in the majority of peroxisomal matrix proteins, and the other is the peroxisomal targeting signal 2 (PTS2) that is located within the N-terminal 30 amino acids of some peroxisomal proteins such as Fox3p. PTS1 consists of the C terminal tripeptide SKL or its conservative variants (S/A/C)(K/R/H)(L/M). Pex5p is the receptor for the PTS 1, whereas importing PTS2-carrying proteins is dependent on Pex7p.

The S. cerevisiae pex5 mutant is viable but accumulates peroxisomal, leaflet-like membrane structures and is deficient in the import of peroxisomal matrix enzymes with a SKL-like import signal such as Fox2p. The acyl-CoA oxidase, Fox1p, follows a novel, non-PTS1, import pathway that is also dependent on Pex5p. In pex5 mutants, both Fox2p and Fox1p are found in the cytosol, but Fox3p is located in the peroxisome.

Activation of fatty acids entering S. cerevisiae can be mediated by at least four different acyl-CoA synthetase gene products. One of these enzymes, Faa2p, is a peroxisomal protein which carries a PTS1 like targeting sequence, while the other three enzymes do not show any obvious peroxisomal targeting sequences. A pex5 mutant is expected to retain the Faa2p in the cytosol enabling cytosolic fatty acid activation.

To test whether S. cerevisiae is able to synthesize increased levels of mc1-PHA in the cytosol, we have expressed the Pseudomonas oleovorans mcl-PHA polymerase in the cytosol of a pex5 receptor mutant.

Strains and Media

Plasmids were maintained and propagated in Escherichia coli DH5α. All S. cerevisiae strains used are described in Example 2. S. cerevisiae BY4743, BY4741-YIL160C and BY4743-YDR244W, which is a heterozygous pex5 mutant strain, were obtained from Invitrogen (Carlsbad, Calif.). Strains wt-16-4 and pex5-16-2 were sporulated from BY4743-YDR244W, and pex5-3c11 was made by mating two haploid pex5 strains using standard protocols (F. Sherman, Methods Enzymol, 350, 3-41 (2002)). S. cerevisiae strains harboring a PHA polymerase gene were grown in SD media (0.67% yeast nitrogen base without amino acids, 2% glucose, and amino acids). For PHA production, a stationary-phase culture grown on glucose was harvested by centrifugation and the cells were washed once in water and resuspended at a 1:10 dilution in fresh SOG1 media containing 0.67% yeast nitrogen base without amino acids, 1% glycerol, 0.4% Tween 80 and the appropriate fatty acids. When cultivating pex5 mutants, cultures were supplemented with geneticin (100 μg/ml). The cultures were then grown on the SOG1 media for 5-6 days before being harvested for PHA analysis. The media utilized either a phosphate (5 mM) or citrate acid (5 mM) buffer to control pH from 4.5 to 7.0.

Cloning Procedure

The plasmids p2TG1T-700(H) and p2TG1T-755(H) are described in Example 1. (FIG. 2)

Analysis of PHA

The cytosolic PHA was studied using gas chromatography-mass spectroscopy analysis, which is described in Example 2.

Results

Cytosolic Expression of the mcl-PHA Polymerase in Wild-Type and Heterozygous pex5 Yeast Strains

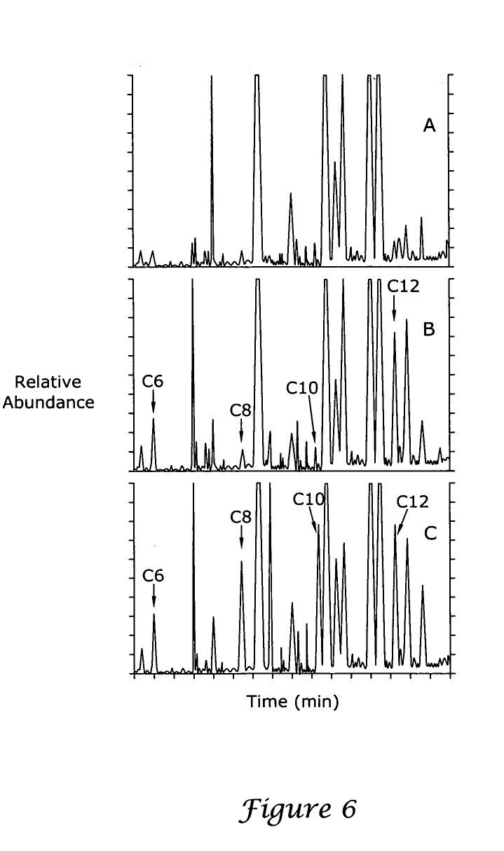

S. cerevisiae strains BY4743, wt-16-4, BY4743-YDR244W, D603, YPH499 and YPH500 were transformed with the PHA polymerase plasmid p2TG1T-700(H). The recombinant yeast was grown in defined medium containing 0.5 g/L lauric acid as the carbon source. The cytosolic expression of the mcl-PHA polymerase resulted in the production of detectable levels of PHA. Cytosolic polymer levels reached approximately 0.015% of the total cell dry weight (CDW) in S. cerevisiae BY4743, while polymer levels reached about 0.026% of the CDW in BY4743-YDR244W, which is 1.7 times higher than in the BY4743 PHA strain. FIG. 6 shows GC-MS analysis of PHA produced by S. cerevisiae BY4743-YDR244W, when lauric acid (C12) was used as the carbon source. FIG. 6A shows the GC-MS trace obtained from a sample of S. cerevisiae BY4743-YDR244W. FIG. 6B shows the GC-MS trace obtained from a sample of S. cerevisiae BY4743-YDR244W harboring p2TG1T-700(H). The C12 (3-hydroxydodecanoic acid) peak, C10 (3-hydroxydecanoic acid), C8 (3-hydroxyoctanoic acid) and C6 (3-hydroxyhexanoic acid) PHA peaks are all clearly visible indicating that these monomers are present in the PHA polymers. The mass to charge ratios of all peaks were checked against PHA produced by an E. coli strain expressing the P. oleovorans PHA polymerase. In the haploid wild-type strain wt-16-4, PHA accumulated to about 0.025% of the CDW.

Yeast strains harboring plasmid p2TG1T-755(H), which expresses a peroxisomally targeted mcl-PHA polymerase, were used as positive controls. Using the same cultivation method, PHA accumulated to 0.042%, 0.053% and 0.054% of the CDW in the peroxisomes of BY4743-YDR244W, BY4743 and wt-16-4 respectively (Table 6 and FIG. 6C). No cytosolic mcl-PHA was detected in wild-type yeast strains D603, YPH499 and YPH500.

Composition of Cytosolic mcl-PHA Produced in Heterozygous pex5 Mutants

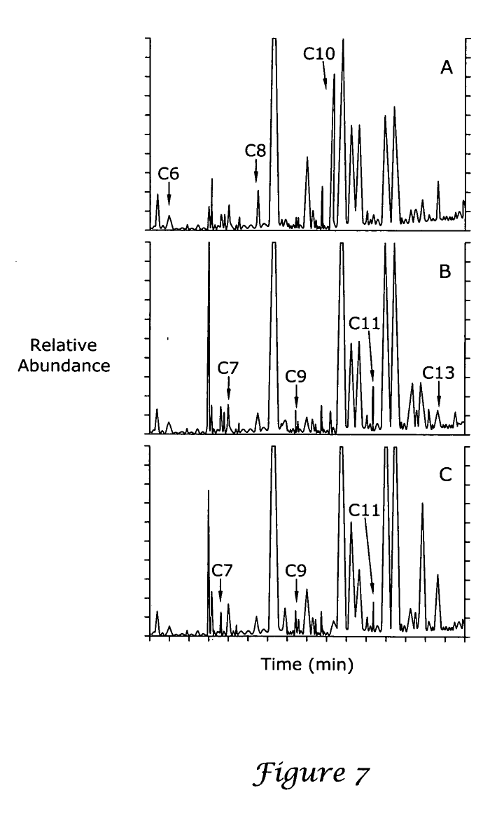

To determine the influence of carbon source on PHA monomer composition, recombinant yeast cells were grown in SOG1 medium containing one of the following fatty acids: oleic acid (C18, 1 g/L), tetradecenoic acid (C14, 0.5 g/L), tridecanoic acid (C13, 0.5 g/L), lauric acid (C12, 0.5 g/L), undecanoic acid (C11, 0.3 g/L) or decanoic acid (C10, 0.3 g/L). The results of the analysis are summarized in Tables 7 and 8. The data demonstrate that the PHA monomer composition is strongly dependent on the externally fed fatty acids (FIG. 7). When C10 fatty acids were used as the carbon source, C10 PHA accounted for about 72% of total biopolymer while no C12 PHA was detected (Table 7). Similarly, recombinant yeast grown on tridecanoic acid (C13) and undecanoic (C11) acid produced PHA containing odd-chain monomers ranging from C13 to C7 with the major monomer components being C13 and C11 PHA (Table 8).

When the recombinant yeast cells were grown in medium containing tetradecenoic acid (C14), only trace amounts of C10, C8 and C6 PHA were detected. No PHA was detected in cultures grown on oleic acid.

Cytosolic mcl-PHA Synthesis in pex5 Mutant Strains

S. cerevisiae pex5-3c11 (homozygous diploid pex5 mutant strain) and pex5-16-2 (haploid pex5 mutant strain) were transformed with plasmids expressing either the PTS1 tagged or nontagged mcl-PHA polymerase (p2TG1T-755(H), p2TG1T-700(H) respectively). These mutant strains can not grow on external fatty acids as the sole carbon source, so the culture media were supplemented with additional glycerol (1-3%, v/w). Lauric acid (0.4 g/L) was used as the carbon source for PHA synthesis. The media were not buffered. After 5-6 days culturing, the cells were harvested and analyzed for PHA.

S. cerevisiae strains pex5-3c11 and pex5-16-2 expressing the mcl-PHA polymerase from plasmid p2TG1T-700(H) accumulated PHA to approximately 0.053% and 0.031 % of their CDW respectively. Similar to the wild-type yeasts, the PHA in the pex5 mutants consisted of C12, C10, C8 and C6 monomers with the C12 monomer representing about 70-85% of the total biopolymer (Table 6). Pex5 mutants harboring p2TG1T-755(H) showed similar results (Table 6).

Composition of Cytosolic mcl-PHA Synthesized in Homozygous pex5 Mutants

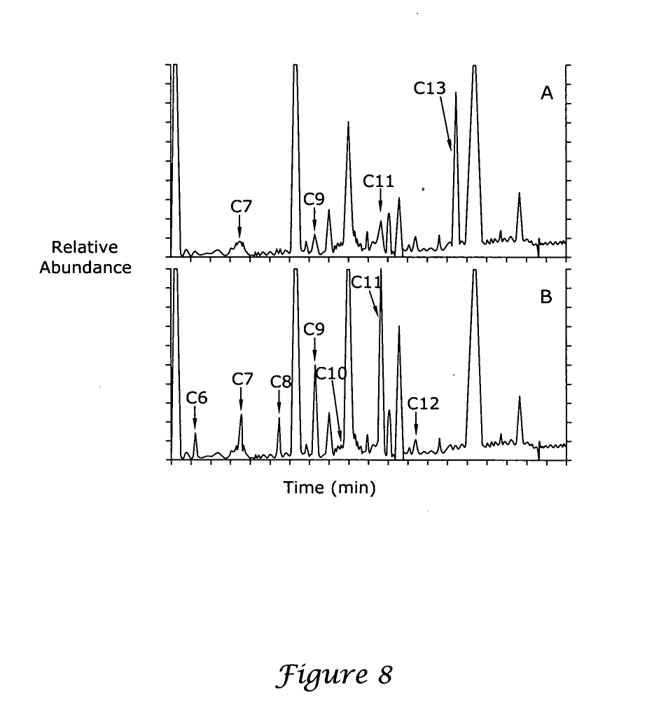

To investigate the influence of the carbon source on PHA monomer composition in pex5 mutants, S. cerevisiae strain pex5-3c11 was grown in SOG1 medium containing either: oleic acid (C18, 0.5 g/L), tridecanoic acid (C13, 0.4 g/L), lauric acid (C12, 0.4 g/L), undecanoic acid (C11, 0.2 g/L) or decanoic acid (C10, 0.2 g/L). Table 5 shows that the PHA monomer composition is dependent on the nature of the external fatty acids. Recombinant yeast grown on C13, C12, C11 and C10 fatty acids, produced PHA comprised primarily of C13, C12, C11 and C10 monomers respectively (FIG. 8). These monomers represent about 45-77% of the total accumulated PHA (Table 9). Interestingly, when undecanoic acid was used as the carbon source, in addition to odd-chain length PHAs, even-chain PHA monomers including C12, C10, C8 and C6 were detected (FIG. 8 and Table 9). These even-chain precursors may originate from fatty acid biosynthesis. When the pex5 mutant was grown in medium containing glycerol and oleic acid or only glycerol, the culture accumulated PHA comprised of C8 and C6 monomers. This also supports the conclusion that fatty acid biosynthesis provides precursors for PHA synthesis in yeast.

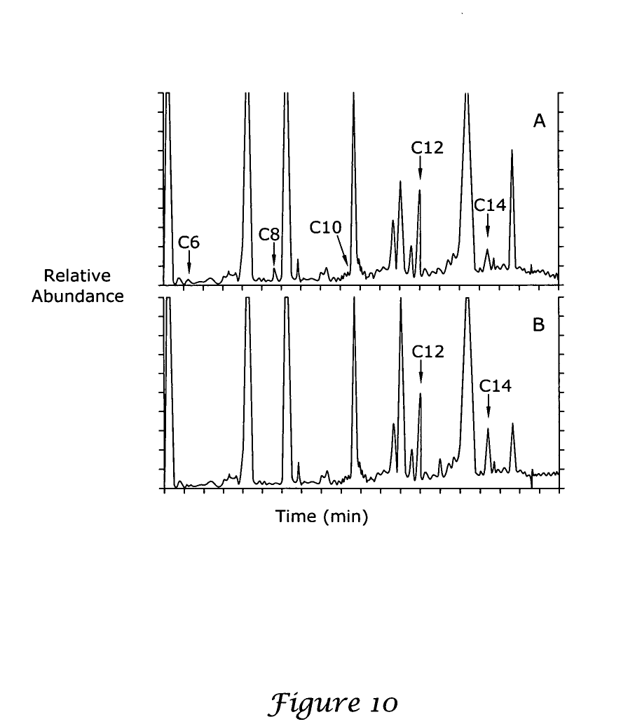

FIG. 10 is GC-MS analysis of PHA produced by S. cerevisiae pex5-3c11 harboring p2TG1T-700, when lauric acid was used as the carbon source. Only peaks, which possess the mass-to-charge ratio value of 131, are shown. S. cerevisiae pex5-3c11 harboring p2TG1T-700 was cultured in pyruvate containing medium (A) and acetate containing medium (B). The arrow indicates the position of PHA monomers.

TABLE 6
mcl-PHA content and monomer composition synthesized
by different yeast strains, when lauric acid (C12) was
used as the carbon source.

| | | | Composition of PHA | | | |
|---|---|---|---|---|---|---|
| | | PHA content | (%, w/w) | | | |
| Hosts | Plasmid | (% of CDW) | C12 | C10 | C8 | C6 |
| BY4743 | p2TG1T-700 | 0.015 ± 0.001 | 58.6 | 16.6 | 22.9 | 1.9 |
| BY4743 | p2TG1T-755 | 0.054 ± 0.004 | 38.7 | 23.8 | 29.9 | 7.6 |
| BY4743- | p2TG1T-700 | 0.026 ± 0.003 | 55.1 | 24.9 | 16.2 | 3.8 |
| YDR244W | | | | | | |
| BY4743- | p2TG1T-755 | 0.042 ± 0.006 | 45.4 | 23.3 | 25.4 | 5.9 |
| YDR244W | | | | | | |
| pex5-3c11 | p2TG1T-700 | 0.053 ± 0.004 | 70.8 | 15.0 | 12.1 | 2.0 |
| pex5-3c11 | p2TG1T-755 | 0.046 ± 0.001 | 81.9 | 5.5 | 10.5 | 2.1 |
| pex5-16-2 | p2TG1T-700 | 0.031 ± 0.009 | 84.8 | 12.3 | 2.0 | 1.0 |
| pex5-16-2 | p2TG1T-755 | 0.028 ± 0.007 | 83.1 | 8.5 | 8.4 | 1.4 |
| wt-16-4 | p2TG1T-700 | 0.025 ± 0.013 | 66.9 | 17.7 | 6.7 | 8.7 |
| wt-16-4 | p2TG1T-755 | 0.054 ± 0.016 | 36.1 | 27.6 | 24.6 | 11.7 |

nd: not detectable

TABLE 7
Cytosolic PHA content and monomer composition produced
by S. cerevisiae BY4743-YDR244W harboring
p2TG1T-700(H) when different even-numbered fatty
acids were fed as the carbon source.

| | | | Composition of PHA | | | |
|---|---|---|---|---|---|---|
| | | PHA content | (%, w/w) | | | |
| Carbon source | | (% of CDW) | C12 | C10 | C8 | C6 |
| C14 | Tetradecanoic | 0.0022 ± 0.0009 | nd | 12.4 | 61.6 | 26.1 |
| | acid | | | | | |
| C12 | Lauric acid | 0.026 ± 0.003 | 55.1 | 24.9 | 16.2 | 3.8 |
| C10 | Decanoic acid | 0.015 ± 0.006 | nd | 72.5 | 23.2 | 4.3 |

nd: not detectable

TABLE 8
Cytosolic PHA content and monomer composition
synthesized by S. cerevisiae BY4743-YDR244W
harboring p2TG1T-700(H) when different odd-
numbered fatty acids were fed as the carbon source.

| | | | Composition of PHA | | | |
|---|---|---|---|---|---|---|
| | | PHA content | (%, w/w) | | | |
| Carbon source | | (% of CDW) | C13 | C11 | C9 | C7 |
| C13 | Tridecanoic acid | 0.017 ± 0.005 | 37.2 | 26.4 | 28.2 | 8.2 |
| C11 | Undecanoic acid | 0.009 ± 0.002 | nd | 38.9 | 30 | 31.1 |

nd: not detectable

TABLE 9
Cytosolic PHA content and monomer composition synthesized
by S. cerevisiae pex5-3c11 harboring p2TG1T-700(H)
when different fatty acids were fed as the carbon source.

| | | Composition of PHA | | | | | | | | |
|---|---|---|---|---|---|---|---|---|---|---|
| | PHA content | (%, w/w) | | | | | | | | |
| Carbon source(s) | (% of CDW) | C14 | C13 | C12 | C11 | C10 | C9 | C8 | C7 | C6 |
| Oleic acid (C18) | 0.0095 ± 0.0039 | | | | | 9.0 | | 57.2 | | 33.8 |
| Tridecanoic acid (C13) | 0.051 ± 0.010 | | 77.4 | | 16.7 | | 3.8 | | 2.1 | |
| Lauric acid (C12) | 0.053 ± 0.004 | | | 70.8 | | 15.0 | | 12.1 | | 2.0 |
| Undecanoic acid (C11) | 0.040 ± 0.005 | | | 0.2 | 46.5 | 0.5 | 24.7 | 13.6 | 7.1 | 7.4 |
| Decanoic acid (C10) | 0.052 ± 0.019 | | | | | 44.6 | | 40.1 | | 15.3 |
| Only glycerol | 0.0013 ± 0.0006 | | | | | | | 82.8 | | 17.2 |

blank: not detectable; all media contain 1-3% glycerol.

Discussion

In this Example, we expressed the P. oleovorans mcl-PHA polymerase in the cytosol of wild-type yeasts and pex5 mutants. The pex5 mutation disrupts the transport of peroxisomal proteins with the PTS1 into the organelle, thus creating a functional cytosolic PHA pathway. The Fox3p enzyme, which possesses a PTS2, is transported into the peroxisomes through the Pex7p transporter (FIG. 5). Expressing a non-targeted P. oleovorans mcl-PHA polymerase in the pex5 mutants permitted the synthesis of mcl-PHA in the cytosol. As shown in Table 6, the pex5 heterozygous yeast strain produced 1.7 times more PHA than the wild-type yeast BY4743 harboring p2TG1T-700(H). This is likely due to a higher concentration of peroxisomal β-oxidation enzymes in the cytosol. The level of cytosolic PHA synthesized by the pex5 mutant is similar to the level synthesized by wild-type yeast expressing a peroxisomally targeted polymerase. Since no PHA was detected in the wild-type yeast strains D603, YPH499 and YPH500, it is believed these strains have mutations in their fatty acids metabolisms.

Wild-type and heterozygous pex5 yeast expressing a cytosolic PHA polymerase did not produce PHA from oleic acid (C18). However, PHA synthesis from oleic acid was observed in strains expressing a peroxisomal polymerase (Example 3). These results, suggest that β-oxidation intermediates can not traverse the peroxisome membrane, and that the non-targeted mcl-PHA polymerase is not transported into the peroxisomes. PHA synthesized by the pex5 mutants from oleic acid contains only C10, C8 and C6 monomers. A possible explanation for why cytosolically expressed polymerase can not produce mcl-PHA from oleic acid is that the degradation of oleic acid, which is an unsaturated fatty acid containing a double bond, occurs via a different pathway than saturated fatty acids.

### Example 5

pH Effect on mcl-PHA Production in a Heterozygous pex5 Mutant

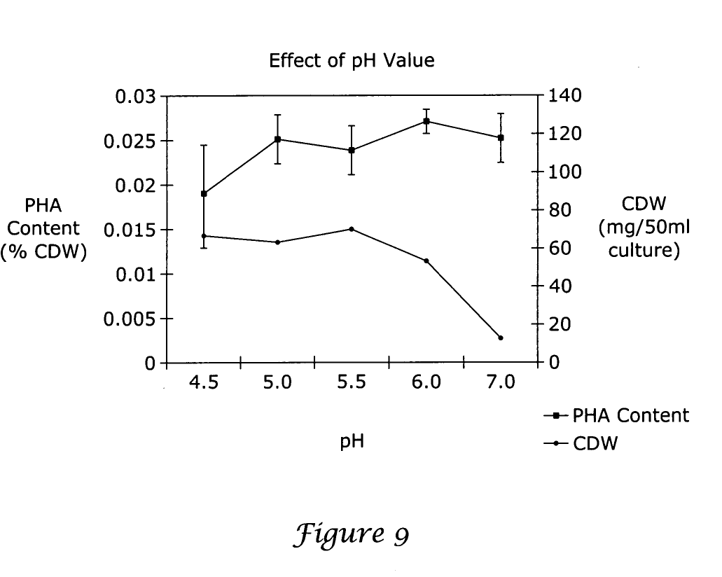

To optimize cultivation condition of S. cerevisiae BY4743-YDR244W harboring p2TG1T-700(H), different phosphate (5 mM) and citric acid (5 mM) buffers were used to control the media pH. The media pH values were varied from 4.5 to 7.0 (FIG. 9). For all pH values, PHA content reached about 0.025% of the cell dry weight however, the CDW was significantly lower for pH values higher than 6.0. When considering high cell viability and PHA production, a pH range of 4.8 to 5.5 was optimal.

### Example 6

Cytosolic mcl-PHA Homopolymer Synthesis in a fox3 Mutant Strain

S. cerevisiae BY4741-YIL160C (haploid fox3 mutant strain) was transformed with plasmids expressing either the PTS1 tagged or nontagged mcl-PHA polymerase (p2TG1T-755(H), p2TG1T-700(H) respectively). This mutant strain can not grow on external fatty acids as the sole carbon source, so the culture media were supplemented with additional glycerol (1-3%, v/w). Lauric acid (0.4 g/L) was used as the carbon source for PHA synthesis. The media were not buffered. After 5-6 days culturing, the cells were harvested and analyzed for PHA.

The haploid fox3 mutant yeast BY4741-YIL160C harboring p2TG1T-700(H) accumulated PHA to about 0.047% of its CDW however the polymer contained only C 12 monomers (homopolymer). When the mcl-PHA polymerase was targeted to the peroxisomes, the yeast accumulated PHA to approximately 0.13% of the CDW. The PHA was comprised of C12, C10, and C8 monomers with the C12 monomers representing the largest fraction (Table 10). The C10 and C8 monomers may have been synthesized by a fatty acid biosynthesis pathway and then degraded by the β-oxidation enzymes in the peroxisomes.

TABLE 10
mcl-PHA content and monomer composition synthesized
by yeast fox3 mutant strains, when lauric acid (C12) was
used as the carbon source.

| | | | Composition of PHA | | | |
|---|---|---|---|---|---|---|
| | | PHA content | (%, w/w) | | | |
| Hosts | Plasmid | (% of CDW) | C12 | C10 | C8 | C6 |
| BY4741- | p2TG1T-700 | 0.047 ± 0.013 | 100.0 | nd | nd | nd |
| YIL160C (fox3) | | | | | | |
| BY4741- | p2TG1T-755 | 0.13 ± 0.05 | 90.1 | 5.9 | 4.0 | nd |
| YIL160C (fox3) | | | | | | |

nd: not detectable

### Example 7

Engineering the Monomer Composition of PHA Synthesized in Yeast

Some factors that could influence PHA synthesis were explored. When the yeast pex5 mutant strains were cultivated in the SOG1 medium containing C12 fatty acid, externally added succinate (5 g/L), malate (1 g/L), oxaloacetate (1 g/L), phosphate (0.5 g/L), serine (1 g/L), glycine (1 g/L), bovine serum albumin (BSA) (0.5 g/L) and NaCl (5 g/L) showed no apparent influence on PHA synthesis. Pyruvate (1 g/L), acetate (0.5 g/L) and formate (0.5 g/L) were tested as alternative carbon sources and tested in an attempt to reduce intracellular coenzyme A concentrations, which is a strong inhibitor of mcl-PHA polymerase.

FIG. 10 is GC-MS analysis of PHA produced by S. cerevisiae pex5-3c11 harboring p2TG1T-700, when lauric acid was used as the carbon source. Only peaks, which possess the mass-to-charge ratio value of 131, are shown. S. cerevisiae pex5-3c11 harboring p2TG1T-700 was cultured in pyruvate containing medium (A) and acetate containing medium (B). The arrow indicates the position of PHA monomers. The use of pyruvate (FIG. 10A), acetate or fornate (FIG. 10B) as carbon sources produced higher final biomass concentrations. In addition, pex5 mutants grown on these substrates accumulated PHA with C14 monomers (Table 11). These C14 PHA precursors were likely synthesized through the fatty acid biosynthesis pathway and degraded in the cytosol by the β-oxidation enzymes that were not transported into the peroxisomes. Homoserine catabolism involves coenzyme A, which is a strong inhibitor of mcl-PHA polymerase. It was added to the media to a final concentration of 0.2% with the hopes of reducing free Coenzyme A levels but it had no significant effect on PHA accumulation.

When the pex5 mutant was grown in medium containing only glycerol, C8 and C6 monomers were detected in the synthesized PHA (Table 11). In addition, if undecanoic acid (C11) was used as the carbon source, the pex5 mutants produced PHA containing some even-chain PHA monomers (Example 4). These even-chain length monomers may have been synthesized by a fatty acid biosynthesis pathway and then degraded by the β-oxidation enzymes in the cytosol. So both external fatty acids and native fatty acids biosynthesis pathways may contribute to the observed PHA synthesis.

TABLE 11
Cytosolic PHA content and monomer composition synthesized
by S. cerevisiae pex5-3c11 harboring p2TG1T-700(H) when
different fatty acids were fed as the carbon source.

| | | Composition of PHA | | | | | | | | |
|---|---|---|---|---|---|---|---|---|---|---|
| | PHA content | (%, w/w) | | | | | | | | |
| Carbon source(s) | (% of CDW) | C14 | C13 | C12 | C11 | C10 | C9 | C8 | C7 | C6 |
| Only glycerol | 0.0013 ± 0.0006 | | | | | | | 82.8 | | 17.2 |
| Homoserine + C12 | 0.050 ± 0.020 | | | 58.4 | | 18.8 | | 18.8 | | 4.5 |
| Pyruvate + C12 | 0.063 ± 0.013 | 1.1 | | 72.7 | | 11.2 | | 11.2 | | 3.8 |
| Acetate + C12 | 0.045 ± 0.007 | 29.1 | | 70.9 | | | | | | |
| Formate + C12 | 0.069 ± 0.002 | 31.9 | | 68.1 | | | | | | |

blank: not detectable; all media contain 1-3% glycerol.

Mcl-PHA was synthesized in either the cytosol or the peroxisome from intermediates of the fatty acid metabolism. The composition of the PHA was strongly influenced by the genetic background of the yeast host, the monomer specificity of the polymerase, the cellular compartment in which the polymerase was active, and the substrate supplied in the medium. The presented data provides a basis for controlling the composition and thus the properties of the synthesized PHA. For instance, homopolymers can be synthesized by the fox3 mutant (BY4741-YIL160C) expressing the cytosolic mcl-PHA polymerase. Polymers of even, odd, or a combination of even and odd numbered monomers can be controlled by feeding the appropriate substrate like fatty acids and glycerol. In addition, the distribution of the monomers can also be influenced by feeding substrates like pyruvate and acetate along with a fatty acid. The presented strategies all hold the potential of creating polymers with novel and desirable material properties.

### Example 8

Strategies for Introducing Multiple mcl-PHA Genes into Yeast

Metabolic pathway engineering often requires the introduction of multiple recombinant genes. Unlike prokaryotes, most eukaryotes do not typically express polycistronic messages. Each gene usually requires its own promoter and its own termination sequence. This makes introduction of multiple genes more difficult.

Two kinds of the expression systems are available for the introduction and regulation of the recombinant, multi-gene PHA pathway in S. cerevisiae. One choice is the GAL1-10 divergent promoter, which permits the co-regulation of two separate genes from a single, centrally located sequence. The single sequence helps reduce plasmid size and does not introduce the possibility of recombination between identical promoters. The divergent GAL1-10 promoter has been used successfully to enhance PHB production in the S. cerevisiae. This was accomplished by regulating a reductase and thiolase from a single bi-directional promoter.

The second choice is a plasmid containing multiple promoters and multiple genes. A tandem gene expression cassette that uses the constitutive GAP promoter needs to be constructed to express the P. oleovorans PHA genes, polymerase, acyl-CoA dehydrogenase, trans-2-enoyl-CoA hydratase and acyl-CoA synthetase.

Vector Constructions

The fadE gene from E. coli that encodes an acyl-CoA dehydrogenase was amplified from the genome of E. coli K12 MG1655 by PCR cloning. The primers used were:

SEQ ID NO.3

5′-GGAATTCATGATGATTTTGAGTATTCTCGCTACGGT-3′
and

SEQ ID NO.4

5′-GGAATTCACGCGGCTTCAACTTTCCGCACTTTCTCCGGC-3′

which created an EcoRI upstream and an EcoRI downstream restriction site. The PCR products were ligated into the pCR-Blunt vector (Invitrogen) and created plasmids pBZ101 and pBZ102.

The fox2 gene was modified by PCR cloning to remove the peroxisomal targeting sequence. The primers used were:

| | |
|---|---|
| 5′-AACTCGAGATGCCTGGAAATTTATCCTTCAAAG-3′ | SEQ ID NO.5 |
| and | |
| 5′-ATCCCGGGTTATTTTGCCTGCGATAGTTTTAC-3′ | SEQ ID NO.6 |

which created an XhoI upstream and a SmaI downstream restriction site. The PCR products were ligated into the pCR-Blunt vector (Invitrogen) and created plasmid pBZ106.

The plasmids pDP306 and p2DP306T had Dam methylation problems at ClaI site. First, a new sequence was designed to eliminate the Dam methylation problem that was associated with the ClaI site. The plasmid pDP306 was used as the template to construct the GAL-10 divergent promoter with new ClaI site. The PCR upstream primer:

| | |
|---|---|
| 5′-TTTGAATTCGGTATCGATTTTTTATTGAATT-3′ | SEQ ID NO.7 |

contained a ClaI site and an EcoRI site. The downstream primer:

| | |
|---|---|
| 5′-CCGGTACAATTCGGGTCGACGTTAACTCTCCTT-3′ | SEQ ID NO.8 |

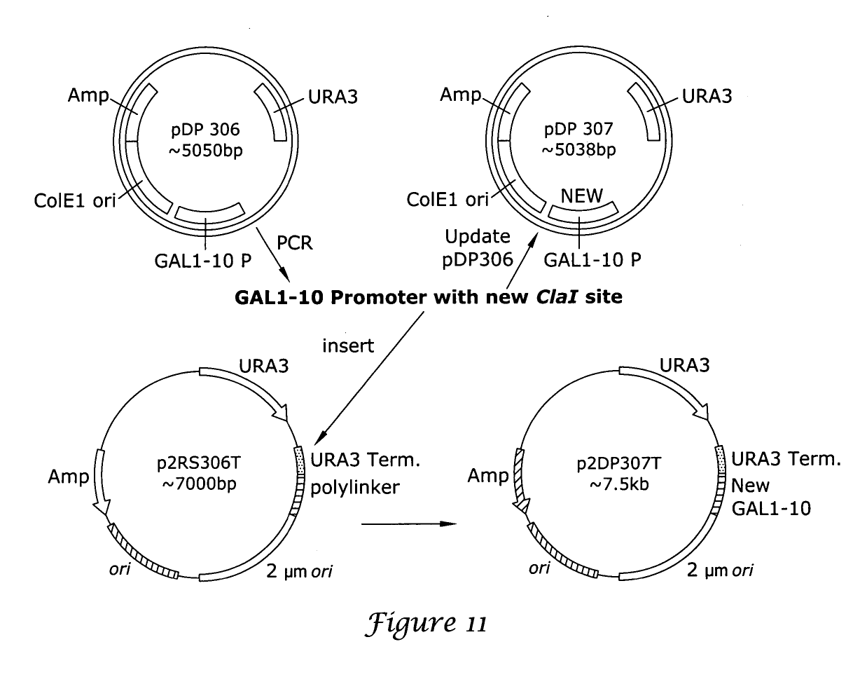

contained a SalI site and a HpaI site. PCR was performed using pfu DNA polymerase (Stratagene) and a Perkin-Elmer PCR thermocycler (30 cycles; melt 95° C. for 45 s, anneal 40° C. for 45 s, extension 72° C. for 120 s). The PCR products were digested with SalI and EcoRI and ligated into similarly-digested plasmid pDP306 and p2RS306T. The resultant plasmids were named pDP307 and p2DP307T, respectively (FIG. 11).

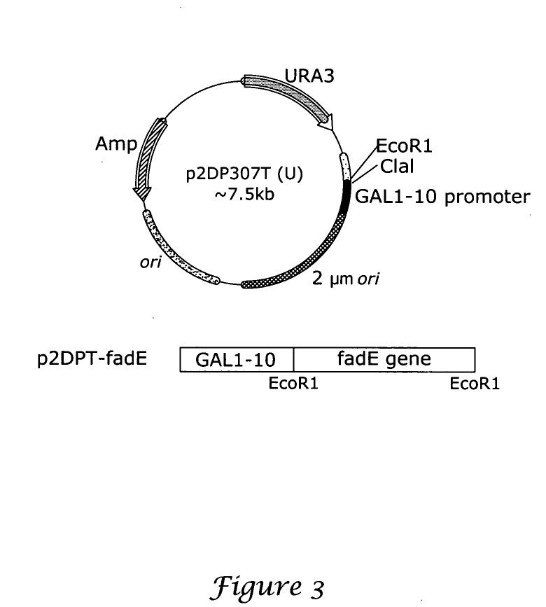

The plasmid p2DP-fadE(U) was created by subcloning the fadE gene into the plasmid p2DP307T using EcoRI digestion. Calf Intestinal Alkaline Phosphatase (CIAP) was used to remove 5′-phosphates from digested p2DP307T to prevent self-ligation during cloning. Plasmid p2DP-fadE(U) is shown in FIG. 3 and carries the 2 μm origin of replication, the new GAL1-10 divergent promoter, the URA3 termination sequence and the E. coli acyl-CoA dehydrogenase gene.

Transformation and Shake Flask Culture

The plasmid p2DP-fadE(U) and p2TEF1-700(H) were co-introduced into the cytosol of S. cerevisiae pex5-3c11 by the lithium acetate procedure (R. Soni et al., Curr Genet. 24, 455-459 (1993)). Transformants were selected on SD medium without uracil and histine. For shake flask experiments, SOG1 medium was used. This medium includes 100 μg/ml Geneticin, 100 mg/L Leucine, 0.67% yeast nitrogen base without amino acids, 1% glycerol, 0.1% yeast extract, 0.4% Tween 80 and the appropriate fatty acids of 0.24 g/L. For PHA production, a stationary-phase culture grown on glucose was harvested by centrifugation and cells were washed once in water and resuspended at a 1:10 dilution in fresh SOG1 medium. To induce the GAL1-10 promoter, cultures were supplemented with the galactose to a final concentration of 0.4%. Cultures were grown on the SOG1 media for 5-6 days before being harvested for PHA analysis. During shake flask studies, all experimental conditions were run in triplicate. The cultures were grown in 250 ml Erlenmeyer flasks containing 50 ml media. The shaker was operated at 200 rpm and 30° C. All reported data is an average of the three separate flake cultures.

S. cerevisiae strains pex5-3c11 harboring p2DP-fadE(U) and p2TEF1-700(H) was grown in defined media containing 0.24 g/L lauric acid as the carbon source. The cytosolic expression of the mcl-PHA polymerase and acyl-CoA dehydrogenase resulted in the production of PHA in the range of about 0.1-0.3% of the CDW or so.

Constitutive Expression System

The constitutive expression system is a plasmid containing multiple constitutive promoters and multiple genes. The plasmid constructed contains the constitutive GAP promoter to express all PHA synthesis genes. The Pichia pastoris vector pGAPZ B was obtained from Invitrogen. The vector pGAPZ B contains following elements: GAP promoter, multiple cloning site with unique restriction sites, C-terminal myc epitope, C-terminal polyhistidine tag, AOX1 Transcription Termination (TT) region, TEF1 promoter, EM7 (synthetic prokaryotic promoter), Sh ble gene (Streptoalloteichus hindustanus ble gene), CYC1 transcription termination region and pUC origin. GAP promoter allows constitutive, high-level expression in Saccharomyce and Pichia. The multiple cloning sites with unique restriction sites allow insertion of the desired gene into the expression vector.

In order to construct a multiple genes expression vector, the BamHI and BglII cassette of the vector pGAPZ B need to be used. BamHI and BglII are two different restriction enzymes and both recognize six base pair DNA targets with the central four bases corresponding to 5′-GATC-3′. If the ends cut by BamHI and BglII were ligated together, both BamHI and BglII sites are inactivated. To construct a vector containing multiple genes expression cassettes, each gene needed to be inserted into the multiple cloning site of pGAPZ-B. It follows that we could utilize the property of the BamHI and BglII cassette to insert the whole cassette into a single plasmid one by one.

The first three enzymes of the fatty acid β-oxidation that are related to the mcl-PHA biosynthesis have BamHI restriction sites in the middle of the gene. The faa2 gene has 2 BamHI sites; the fadE gene has one BamHI site; and the fox2 gene has one BamHI site. Therefore, before cloning the gene into pGAPZ-B, all BamHI sites have to be removed. The technique of site-directed mutagenesis was used.

In vitro site-directed mutagenesis is an invaluable technique for characterizing the dynamic, complex relationships between protein structure and function, for studying gene expression elements, and for carrying out vector modification. The site-directed mutagenesis kit (Stratagene, La Jolla, Calif.), which was used in this example, allows site specific mutation in virtually any double-stranded plasmid, thus eliminating the need for subcloning and for ssDNA rescue. In addition, the site-directed mutagenesis does not require specialized vectors, unique restriction sites, multiple transformations or in vitro methylation treatment steps.

The primers used to mutate the fadE gene were:

| | |
|---|---|
| 5′-CCGGCGTGAGCGGAATCCTGGCGATTA-3′ | SEQ ID NO.9 |
| and | |
| 5′-TAATCGCCAGGATTCCGCTCACGCCGG-3′ | SEQ ID NO.10 |

that replace the original codon GGG with GGA to remove the BamHI site. The primers used to mutate fox2 gene were:

SEQ ID NO.11

5′-AAGGTAGTTGTAAATGACATCAAGGACCCTTTTTCAGTTGTTGAAGA
AATA-3′
and

SEQ ID NO.12

5′-TATTTCTTCAACAACTGAAAAAGGGTCCTTGATGTCATTTACAACTA
CCTT-3′

which replace the original codon GAT with GAC to remove the BamHI site. All primers are 5′ phosphorylated and purified by polyacrylamide gel electrophoresis (PAGE).

To construct a vector containing multiple PHA genes expression cassettes, faa2, fadE and fox2 genes needed to be inserted into the multiple cloning site of pGAPZ-B. It follows that we could utilize the property of the BamHI and BglII cassette to insert the whole cassette into a single plasmid one by one. A 2 μm replication origin of Saccharomyce was inserted this plasmid to create a single yeast plasmid that containing multiple PHA synthesis genes.

### Example 9

Sc1-PHA Production in Recombinant Yeast

Cloning Procedure

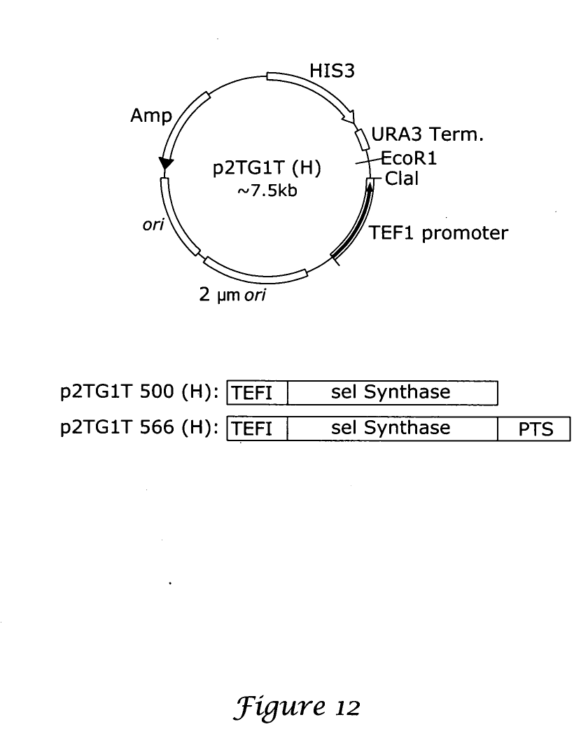

The Ralstonia eutropha scl-PHA synthase gene was isolated from plasmid pPT 500 (Jackson, Recombinant Modulation of the phbCAB Operon Copy Number in Ralstonia eutropha and Modification of the Precursor Selectivity of the Pseudomonas oleovorans Polymerase I. Masters Dissertation. University of Minnesota. St. Paul, Minn., (1998)) using ClaI and EcoRI, and ligated into similarly digested p2TG1T(H). The resulting plasmid was named p2TG1T-500(H) and is depicted in FIG. 12. The plasmid p2TG1T-500(H) contains the TEF1 promoter, 2 μm origin, HIS3 marker and URA3 terminal sequence and expresses the R. eutropha scl-PHA synthase. R. eutropha scl-PHA synthase containing the peroxisomal targeting sequence was obtained by PCR-cloning of p2TG1T-500(H). The primers used were:

SEQ ID NO.13

5′-ATTATCGATGGCGACCGGCAAAGGCGCGGC-3′
and

SEQ ID NO.14

5′-GGAATTCACAATCTAGCCACAGCTCTTGCCTTGGCTTTGACGT
AT-3′

The 3′ primer modified the phbC gene by adding a six amino acid peptide (ARVARL) to the 3′ end, which was shown by J. J.Hahn et al., Biotechnol Prog, 15, 1053-1057 (1999) to target scl-PHA synthase to the peroxisome of maize. The PCR product was digested with ClaI and EcoRI, and ligated into a similarly digested p2TG1T(H) to create p2TG1T-566(H) as depicted in FIG. 12. The plasmid p2TG1T-566(H) contains R. eutropha scl-PHA synthase with peroxisomal targeting sequence (PTS). The plasmids were transferred into the S. cerevisiae strains using the lithium acetate procedure.

Ralstonia Eutropha scl-PHA Synthase Expression in the Cytosol and in Peroxisomes

S. cerevisiae strain BY4743 was transformed with either the nontargeted or targeted PHA synthase plasmid (p2TG1T-500(H) or p2TG1T-566(H) respectively). The recombinant yeasts were grown in defined medium containing oleic acid (1 g/L) as the carbon source. The cytosolic expression of the scl-PHA synthase resulted in the synthesis of PHA, which accumulated to 0.02% of the CDW. In the strain expressing the peroxisomally targeted enzyme, the PHA content was approximately 0.8% of the CDW.

The carbon source was varied to test the effect on monomer composition of peroxisomally-produced PHA. The recombinant yeasts were grown on SOG1 medium containing one of the following fatty acids: lauric acid (C12, 0.5 g/L), tridecanoic acid (C13, 0.5 g/L) and a mixture of 0.25 g/L lauric acid and 0.25 g/L tridecanoic acid. The results are summarized in Table 12. When the peroxisomally targeted synthase strain was fed even-chain fatty acids, the accumulated PHA was comprised of approximately 97-99% C4 monomers, with the balance being C8, C6 and C5 monomers. Similarly, feeding an odd number C13 fatty acid resulted in a PHA copolymer comprised of approximately 6% C4 and 94% C5 monomers. When the yeasts were fed a mixture of C12 and C13 fatty acids, polymer levels reached approximately 7% of the CDW. The peroxisomally synthesized PHA was comprised of 84% C4 and 16% C5 monomers, with the balance being C6 and C8 monomers.

TABLE 12
Peroxisomal PHA content and monomer composition
synthesized by S. cerevisiae BY4743 harboring p2TG1T-566(H)
when different fatty acids were fed as the carbon source.

| | | | Composition of PHA | | | |
|---|---|---|---|---|---|---|
| | | PHA content | (%, w/w) | | | |
| Carbon source | | (% of CDW) | C4 | C5 | C6 | C8 |
| C18 | Oleic acid | 0.8 ± 0.04 | 97.2 ± 0.5 | 1.4 ± 0.4 | 1.2 ± 0.2 | 0.2 ± 0.1 |
| C12 | Lauric acid | 3.8 ± 0.1 | 98.9 ± 0.3 | 0.18 ± 0.03 | 0.6 ± 0.2 | 0.3 ± 0.1 |
| C13 | Tridecanoic acid | 1.6 ± 0.1 | 6 ± 1 | 94 ± 1 | nd | nd |
| C12 and | Lauric acid & | 6.9 ± 0.1 | 84 ± 2 | 16 ± 1 | 0.3 ± 0.1 | 0.2 ± 0.1 |
| C13 | Tridecanoic acid | | | | | |

nd: not detectable

S. cerevisiae strains harboring the scl-PHA synthase from R. eutropha produced PHA in the peroxisomes up to 7% of the cell dry weight. The scl-PHA was comprised of C8-C4 monomers. The results confirm those obtained by V. C. De Oliveira et al. (Appl Environ Microbiol 70:5685-5687, (2004)); however, the polymer levels in our example are about 100 times higher than in the previous study. The difference could be a result of using a different yeast strain, an improved promoter system or different medium.

### Example 10

Preparing Yeast Hosts for PHA production

Yeast Strains

Saccharomyces Cerevisiae strain BY4743-YDR244W (Mata/α his3Δ1 leu2Δ0 ura3Δ0 pex5::kanMX4), which is a heterozygous pex5 diploid yeast strain, was used to prepare suitable hosts for production of PHA.

Media

KAC medium (Potassium acetate 1%, Yeast extract 0.1%), KAC 100K medium (Peptone 2%, Yeast Extract 1%, KCl 0.75%) and YPD 100K medium(Glucose 2%, Peptone 2%, Yeast Extract 1%, KCl 0.75%) were used.

Sporulation

Under the appropriate environmental conditions, a diploid yeast cell will undergo meiosis, separate all their chromosomes into haploid sets once more, and package the results into four, smaller, separate haploid cells which can be clearly seen and micro-manipulated under a microscope. The tiny cluster of four haploid cells (called “spores”) that results from a single round of meiotic division stay together in a structure called an ascus. This makes it possible for the experimenter to identify them as a tetrad of sibling spores. After suitable enzymatic digestion of the ascus wall, each spore can be teased apart using a very fine glass probe. Each of the four haploids cells can then be grown into independent colonies of identical cells that can be studied for the genes they carry.

S. cerevisiae diploid strain BY4743-YDR244W was placed on KAC plates, which have a low concentration of nitrogen. This causes the diploid to sporulate, or go through meiosis, and forms a tetrad of haploid spores. When attempting to sporulate a yeast strain, transfer the diploid from YPD 100K to KAC. Streak the yeast very thinly for best results. Leave the plate on the desktop for three days and then transfer it to the incubator for 24 hours. After 24 hours, return it to the desktop and it will be ready to dissect.

Digestion and Dissection

The resulting tetrads of haploid spores need to be dissected and analyzed. The first step in the dissection process is the digestion of the ascus surrounding the tetrad. In the hood, place 50 μl of the 1:10 of 10:40 dilution in sorbitol of stock lytic enzyme in a microtube. Second, use a sterile toothpick to obtain sporulated yeast from a KAC plate. Third, place the toothpick in the 50 μl of lytic enzyme in the microtube and swirl the toothpick for about 30 seconds. Finally, allow the enzyme to digest for another 30 seconds and then add 1 ml of sterile water to inactivate the enzyme. Obtain a YPD 100K dissection plate; draw a line using a black marker and a ruler; sterilize an inoculating loop by passing it through the flame of a Bunsen burner. Stick the sterile loop in the solution of digested yeast and streak it on the dissection plate along the black line. Repeat this step about four times. Invert the plate and place it in the ring on the stage of the micromanipulator. Position the stage so that the needle is in the large open area of the plate away from any cells. Use the lowest magnification lens and try to find the needle. Next, look in the microscope and move the joystick around until you see the needle. Use the fine focus if necessary. The agar is covered with a very thin film of water, and when the needle touches this film, a dark ring will be seen around the needle. Once the needle was found, raise it using the joystick, reposition the stage, and start searching for tetrads. Look for groups of four spores and pick them up with the micromanipulator needle. Move the stage so that the spores can be placed on the large empty portion of the plate. Twelve tetrads will fit on one plate.

Making Crosses

To make a cross, place a small amount of the two haploid strains of opposite mating type that you wish to cross approximately 1 cm apart on a YPD plate. Next, place about 20 μl of water between them. Next, take a sterile toothpick and combine the two haploid strains and swirl them around in the water. Place the plate in a plastic bag, put it in the 30° C. incubator, and return four hours later to pick the resulting diploids.

Resulting yeast hosts that are available for PHA synthesis pathway expression are listed in Table 13. For instance, yeast pex5-3c11 was employed in Example 4.

TABLE 13
List of Yeast Strains from Sporulation

| Name | Genotype |
|---|---|
| wt-3-1 | Mata his3Δ1 leu2Δ0 ura3Δ0 met15Δ0 |
| wt-3-2 | Matα his3Δ1 leu2Δ0 ura3Δ0 |
| wt-8-3 | Mata his3Δ1 leu2Δ0 ura3Δ0 met15Δ0 |
| wt-8-4 | Mata his3Δ1 leu2Δ0 ura3Δ0 lys2Δ0 met15Δ0 |
| wt-10-1 | Matα his3Δ1 leu2Δ0 ura3Δ0 |
| wt-10-2 | Mata his3Δ1 leu2Δ0 ura3Δ0 lys2Δ0 met15Δ0 |
| wt-11-3 | Matα his3Δ1 leu2Δ0 ura3Δ0 |
| wt-11-4 | Mata his3Δ1 leu2Δ0 ura3Δ0 lys2Δ0 |
| wt-12-2 | Mata his3Δ1 leu2Δ0 ura3Δ0 met15Δ0 |
| wt-12-4 | Matα his3Δ1 leu2Δ0 ura3Δ0 lys2Δ0 |
| wt-16-3 | Matα his3Δ1 leu2Δ0 ura3Δ0 met15Δ0 |
| wt-16-4 | Matα his3Δ1 leu2Δ0 ura3Δ0 lys2Δ0 |
| pex5-3-3 | Matα his3Δ1 leu2Δ0 ura3Δ0 met15Δ0 lys2Δ0 pex5::kanMX4 |
| pex5-3-4 | Mata his3Δ1 leu2Δ0 ura3Δ0 lys2Δ0 pex5::kanMX4 |
| pex5-8-1 | Matα his3Δ1 leu2Δ0 ura3Δ0 pex5::kanMX4 |
| pex5-8-2 | Matα his3Δ1 leu2Δ0 ura3Δ0 lys2Δ0 pex5::kanMX4 |
| pex5-10-3 | Mata his3Δ1 leu2Δ0 ura3Δ0 lys2Δ0 pex5::kanMX4 |
| pex5-10-4 | Matα his3Δ1 leu2Δ0 ura3Δ0 met15Δ0 pex5::kanMX4 |
| pex5-11-1 | Mata his3Δ1 leu2Δ0 ura3Δ0 met15Δ0 lys2Δ0 pex5::kanMX4 |
| pex5-11-2 | Matα his3Δ1 leu2Δ0 ura3Δ0 met15Δ0 pex5::kanMX4 |
| pex5-12-3 | Matα his3Δ1 leu2Δ0 ura3Δ0 met15Δ0 pex5::kanMX4 |
| pex5- | Mata/α his3Δ1 leu2Δ0 ura3Δ0 pex5::kanMX4 |
| 3c11¹ | |
| pex5- | Mata/α his3Δ1 leu2Δ0 ura3Δ0 pex5::kanMX4 |
| 10c11² | |
| pex5-16-1 | Mata his3Δ1 leu2Δ0 ura3Δ0 pex5::kanMX4 |
| pex5-16-2 | Mata his3Δ1 leu2Δ0 ura3Δ0 lys2Δ0 met15Δ0 pex5::kanMX4 |

¹pex5-3c11 was obtained by mating pex5-3-4 and pex5-11-2.
²pex5-10c11 was obtained by mating pex5-10-3 and pex5-11-2

### Example 11

Developing Yeast Culture Strategies for PHA Production

Yeast Strains

Saccharomyces cerevisiae strain D603 (MATa/MATα ura3-52 lys2-801 met his3 ade2-101 regl-501) was used as the host strain.

Media and Culture Conditions

Wild type S. cerevisiae cultures were grown on YPD medium. Minimal medium contained 0.67% yeast nitrogen base without amino acids (YNB) (Difco Laboratories, Detroit, Mich.) and amino acids (20 μg/ml) as needed, and supplemented with 2% glucose (SD) or other carbon sources. All media are listed in Table 14. Yeast cells were grown on plates or in Erlenmeyer flasks at 30° C.

TABLE 14
Media used in this and other examples.

| Medium Name | Composition |
|---|---|
| YPD | 1% yeast extract, 2% peptone and 2% glucose |
| YP | 1% yeast extract and 2% peptone |
| SD* | 0.67% YNB¹, amino acids and 2% glucose |
| SG* | 0.67% YNB, amino acids, 0.1% yeast extract and 2% |
| | glycerol |
| SO* | 0.67% YNB, amino acids, 0.2% oleic acid and 0.2% |
| | Tween 80 |
| SOD* | SO plus 0.1% glucose |
| SOY* | SO plus 0.1% yeast extract |
| SOM* | SO plus 0.1% yeast extract and 0.5% maltose |
| SOG1* | SO plus 0.1% yeast extract and 1% glycerol |
| SOG2* | SO plus 0.1% glycerol |
| SOG3* | SO plus 0.1% yeast extract and 0.1% glycerol |

*The medium contains 0.5% potassium phosphate buffer, pH 6.8.
¹YNB: yeast nitrogen base.

Plasmids and Expression

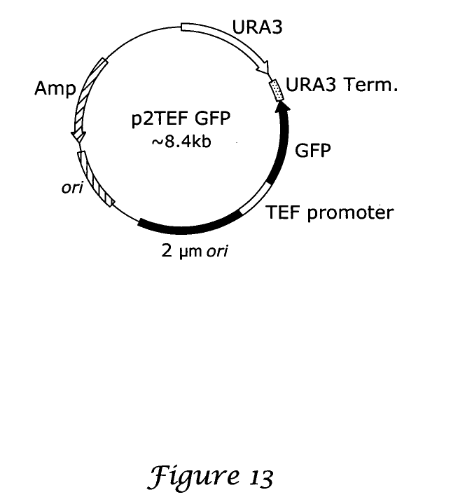

The plasmid p2TEF-GFP containing TEFI promoter, 2 μm origin, URA3 marker and URA3 terminal sequence expressed green fluorescence protein (Gfp) in S. cerevisiae (FIG. 13).

Flow Cytometry

A Becton-Dickinson FACSCalibur flow cytometer (Becton-Dickinson Immunocytometry System, San Jose, Calif.) with a 15-mW Ar laser with a wavelength of 488 nm was utilized to determine the fluorescence intensity of the cells containing Gfp. QuicKeys software (CE Software, West Des Moines, Iowa) was used to control the proprietary Cell Quest software (Becton Dickinson, San Jose, Calif.) on a Macintosh computer for data acquisition from the FACSCalibur flow cytometer. Gfp fluorescence was determined using a 530±30 band pass filter. The data was collected using logarithmic amplification.

Fluorescent Dyes and Microscope

Propidium iodide (PI): PI stains DNA and is known as an exclusion dye, which only stains dead cells that are lacking membrane integrity therefore allowing the dye into the cytoplasm. Using the cell count data, each sample was diluted to 5×10⁶ in 1 ml PBS containing 10 μg/ml PI. This concentration will allow 1000 cells/s, which is the optimal event rate, to be run on the flow cytometer on the low setting (M. AlRubeai et al., “A rapid method for evaluation of cell number and viability by flow cytometry,” Cytotechnology, 24, 161-168 (1997)). Nile red and bodipy 493/503 are able to selectively stain intracellular lipids. Yeast cells were stained using Nile red and bodipy, then observed using Nikon Eclipse E800 microscope, equipped with phase, DIC, darkfield and epi-fluorescence capabilities.

Dynamics

Because S. cerevisiae grows poorly on fatty acids, most of the culture time is in the stationary phase and the death phase. Therefore, the concentration and the number of cells are given by the following equations:

For stationary phase

$$\frac{ⅆ {C}_{x}}{ⅆ t} = ( μ - {k}_{d} ) {C}_{x} = 0 , \\ \frac{ⅆ {N}_{x}}{ⅆ t} = ( μ - {k}_{d} ) {N}_{x} = 0 ,$$

For death phase

$$\frac{ⅆ {C}_{x}}{ⅆ t} = - {k}_{d} {C}_{x} , \\ {C}_{x} = {C}_{0} exp ( - {k}_{d} t ) , \\ {N}_{x} = {N}_{0} exp ( - {k}_{d} t ) ,$$

where C denotes the concentration of cells, N denotes the number of cells, μ denotes specific growth rate, and k_{d} denotes death rate constant.

Growth in Oleic Acid Only Medium

S. cerevisiae cells were grown on SD medium for 24 hours, then shifted into SO medium and cultured for 144 hours. The samples were stained using propidium iodide (PI), then examined using flow cytometer to determine cell viability.

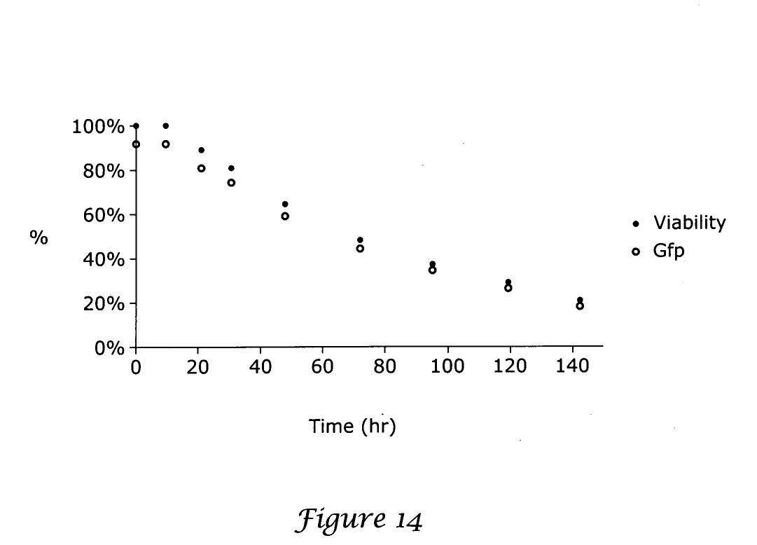

Initially, 98.6% of yeast cells were viable and 89.6% of cells showed green fluorescence. After 72 hours, half of the cells were dead. At the end of 6-day culture, only 22.6% of cells were still alive and 21.1% of cells were viable and showed fluorescence (FIG. 14). The death rate constant k_{d} was 0.0108. The poor growth of S. cerevisiae on oleic acids was quantitatively determined.

Different Components were Added to Help the Growth of S. cerevisiae on Oleic Acid

Currently, glucose and yeast extract are widely used to help S. cerevisiae cells grow in fatty acid medium. Also it is reported that glycerol and maltose do not repress the β-oxidation system of S. cerevisiae, and they can only function with yeast extract. Therefore four kinds of media including above components were examined: 1) SOD, 2) SOY, 3) SOG1, and 4) SOM (Table 7-1).

By flow cytometry analysis, both 0.1% glucose and 0.1% yeast extract helped the growth of S. cerevisiae in oleic acid medium. After a 6-day culture in SOD medium, 47.0% of the cells survived, 43.3% of the cells were viable and kept fluorescence and kd was 0.0059. At the end of cultivation in SOY medium, 50.0% of the cells were alive, and 44.1% of the cells were viable and contained Gfp compared to 21.1% in SO medium. The death rate constant k_{d} was 0.0053 in SOY medium.

Because the uptake of glycerol was very quick in SOG1 medium, OD₆₀₀ increased from 1.0 to 2.3 in first 30 hours. From flow cytometry data, some large size yeast cells showing green fluorescence were observed after 24 hours culture. But as glycerol was depleted, the death of cells was fast. The death rate constant was 0.0041, 57.8% of the cells survived, and 51.6% of the cells were viable and contained Gfp. Maltose was consumed gradually during 144 hours culture, this also helped the growth of yeast in SOM medium, 54.1% of the cells were viable after 6 days culture, and 49.3% of the cells were viable and contained Gfp. The death rate constant was 0.0046.

Glucose Free Culture

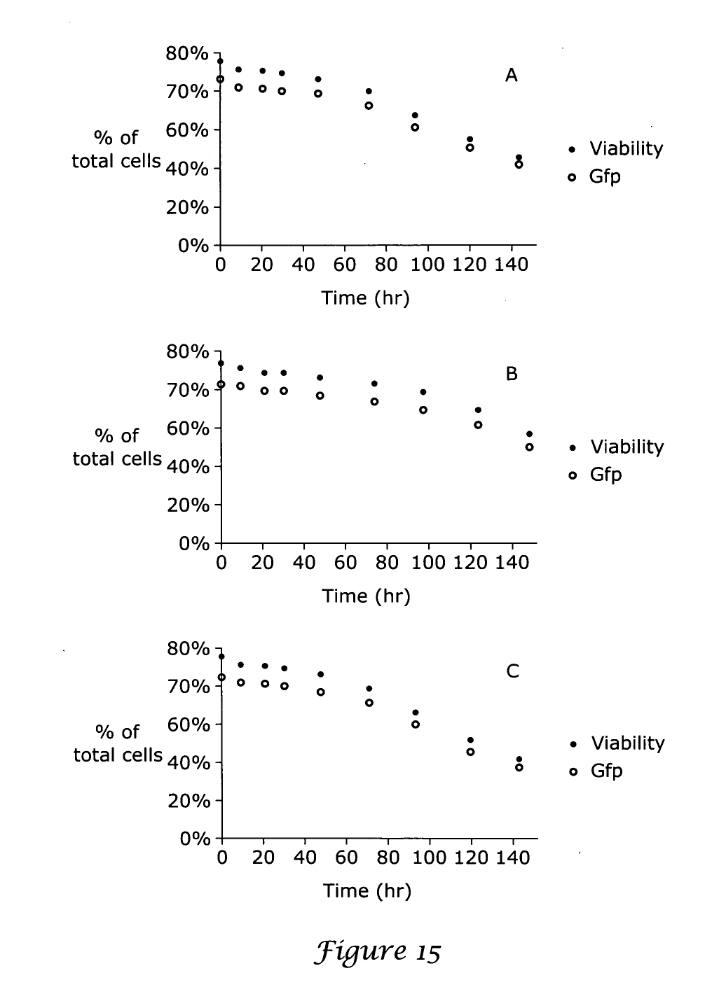

A “boosted” strategy was examined. After pre-cultured in SG, the culture was boosted in YP medium for 4 hours. Then, the cells were harvested, shifted to SOY, SOG3 and SOM media, and cultured for 120-144 hours. At the end of a 120 hours culture, the viability of yeast cells in the three media were 44.0%, 56.9% and 66.6%, respectively; and 40.7%, 50.1% and 60.3% of cells were viable and showed green fluorescence respectively (FIG. 15). The death rate constants were 0.0055, 0.0030 and 0.0029 respectively.

Discussion

In the present example, a cultivation strategy (SG to YP to SOG3) was developed using flow cytometry. The maltose medium is not recommended because the consumption of maltose is slow, and this represses the consuming of other carbon sources, such as galactose. When the inducible GAL1-10 promoter was used to express genes in S. cerevisiae, maltose strongly inhibited the Gal promoter activity. If the constitutive promoter is used, maltose is a good choice.

When S. cerevisiae D603 harboring Gfp was cultivated in SD medium, about 10% of cells did not show green fluorescence. The reason may be the loss of the plasmid. After yeast cells were shifted from YP medium to oleic acid medium, approximately 5% of cells died in 10 hours. The possible reason is that YP medium is non-selective, both wild type and recombinant yeast cells can grow in it. So wild-type yeast cells will die fast after shifting into oleic acid medium with the selective pressure.

Poor growth of S. cerevisiae on oleic acid or other fatty acids limits the ability to produce β-oxidation related products, such as PHA. With the developed culture strategy, S. cerevisiae may produce higher amounts of β-oxidation related products. A combination of flow cytometry technology and the expression of green fluorescent protein permit a quantitative and quick analysis of the physiology of S. cerevisiae. This combination also permits us to quickly optimize culture conditions to promote PHA production in yeast strains.

What is claimed is:

1. A transgenic microorganism, comprising:
a yeast strain including a heteologous nucleic acid that operably encodes a polyhydroxyalkanoate polymerase.

2. The transgenic microorganism claim 1, wherein the yeast strain is from the genera Saccharomyces.

3. The transgenic microorganism claim 1, wherein the yeast strain is a wild type yeast strain transfected with the heteologous nucleic acid that operably encodes a polyhydroxyalkanoate polymerase.

4. The transgenic microorganism claim 1, wherein the yeast strain includes a mutation of one or more genes selected from the group comprising pex5, pex7, pex8, pex13, pex14, pex18, pex21, and fox3, and wherein the yeast strain is transfected with the heteologous nucleic acid that operably encodes a polyhydroxyalkanoate polymerase.

5. The transgenic microorganism of claim 1, wherein the polyhydroxyalkanoate polymerase produces polyhydroxyalkanoate in the cytosol of the yeast strain.

6. The transgenic microorganism of claim 1, wherein the yeast strain lacks at least one naturally occurring peroxisomal targeting sequence receptor protein.

7. The transgenic microorganism of claim 1, wherein the polyhydroxyalkanoate polymerase is a short chain length polyhydroxyalkanoate polymerase.

8. The transgenic microorganism of claim 1, wherein the polyhydroxyalkanoate polymerase is a medium chain length polyhydroxyalkanoate polymerase.

9. The transgenic microorganism of claim 1, wherein the polyhydroxyalkanoate polymerase is a peroxisomally-targeted polyhydroxyalkanoate polymerase.

10. The transgenic microorganism of claim 1, wherein the polyhydroxyalkanoate polymerase is encoded by a plasmid.

11. A method for producing polyhydroxyalkanoate in a microorganism, comprising the steps of:
providing a yeast strain, the yeast strain including a heteologous nucleic acid that operably encodes a polyhydroxyalkanoate polymerase;
supplying a carbon source to the yeast strain;
culturing the yeast strain so that polyhydroxyalkanoate is produced; and
isolating the polyhydroxyalkanoate from the yeast strain.

12. The method of claim 11, wherein the step of providing a yeast strain includes providing a wild type yeast strain.

13. The method of claim 12, further comprising the step of transfecting the wild type yeast strain with a vector comprising the heterologous nucleic acid.

14. The method of claim 11, wherein the step of providing a yeast strain includes providing a yeast strain that includes a mutation of one or more genes selected from the group comprising pex5, pex7, pex8, pex13, pex14, pex18, pex21, and fox3.

15. The method of claim 14, further comprising the step of transfecting the yeast strain with a vector comprising the heterologous nucleic acid.

16. The method of claim 11, wherein the step of culturing the yeast strain so that polyhydroxyalkanoate is produced includes producing polyhydroxyalkanoate in the cytosol of the yeast strain.

17. The method of claim 11, wherein the polyhydroxyalkanoate polymerase is a medium chain length polyhydroxyalkanoate polymerase.

18. The method of claim 11, wherein the polyhydroxyalkanoate polymerase is a short chain length polyhydroxyalkanoate polymerase.

19. The method of claim 11, wherein the polyhydroxyalkanoate polymerase is a peroxisomally-targeted polyhydroxyalkanoate polymerase.

20. A non-human eukaryotic organism, comprising:
a eukaryotic organism including a heteologous nucleic acid that operably encodes a polyhydroxyalkanoate polymerase; and
wherein the eukaryotic organism is a transgenic microorganism.


SEQUENCE LISTING (14 sequences)

SEQ ID NO: 1 - DNA, 31 bp, Artificial
  synthetic oligonucleotide
attatcgatgagtaacaagaacaacgatgag

SEQ ID NO: 2 - DNA, 34 bp, Artificial
  synthetic oligonucleotide
ggaattcatagcttggaacgctcgtgaacgtagg

SEQ ID NO: 3 - DNA, 36 bp, Artificial
  synthetic oligonucleotide
ggaattcatgatgattttgagtattctcgctacggt

SEQ ID NO: 4 - DNA, 39 bp, Artificial
  synthetic oligonucleotide
ggaattcacgcggcttcaactttccgcactttctccggc

SEQ ID NO: 5 - DNA, 33 bp, Artificial
  synthetic oligonucleotide
aactcgagatgcctggaaatttatccttcaaag

SEQ ID NO: 6 - DNA, 32 bp, Artificial
  synthetic oligonucleotide
atcccgggttattttgcctgcgatagttttac

SEQ ID NO: 7 - DNA, 31 bp, Artificial
  synthetic oligonucleotide
tttgaattcggtatcgattttttattgaatt

SEQ ID NO: 8 - DNA, 33 bp, Artificial
  synthetic oligonucleotide
ccggtacaattcgggtcgacgttaactctcctt

SEQ ID NO: 9 - DNA, 27 bp, Artificial
  synthetic oligonucleotide
ccggcgtgagcggaatcctggcgatta

SEQ ID NO: 10 - DNA, 27 bp, Artificial
  synthetic oligonucleotide
taatcgccaggattccgctcacgccgg

SEQ ID NO: 11 - DNA, 51 bp, Artificial
  synthetic oligonucleotide
aaggtagttgtaaatgacatcaaggaccctttttcagttgttgaagaaata

SEQ ID NO: 12 - DNA, 51 bp, Artificial
  synthetic oligonucleotide
tatttcttcaacaactgaaaaagggtccttgatgtcatttacaactacctt

SEQ ID NO: 13 - DNA, 30 bp, Artificial
  synthetic oligonucleotide
attatcgatggcgaccggcaaaggcgcggc

SEQ ID NO: 14 - DNA, 45 bp, Artificial
  synthetic oligonucleotide
ggaattcacaatctagccacagctcttgccttggctttgacgtat